(19) United States
(12) Patent Application Publication
(10) Pub. No.: US 2025/0108085 A1
(43) Pub. Date: Apr. 3, 2025

(54) COMPOSITIONS COMPRISING HEMP EXTRACT AND RELATED TEATMENT METHODS

(71) Applicant: Portland Technology Holdings LLC, South Portland, ME (US)

(72) Inventors: Christian Kjaer, South Portland, ME (US); Joseph Wakshlag, South Portland, ME (US)

(21) Appl. No.: 18/829,965

(22) Filed: Sep. 10, 2024

Related U.S. Application Data
(63) Continuation of application No. PCT/US2023/064095, filed on Mar. 10, 2023.
(60) Provisional application No. 63/269,127, filed on Mar. 10, 2022.

(51) Int. Cl. A61K 36/185; A61K 31/00; A61P 1/00; A61P 25/04; A61P 25/22

(52) U.S. Cl. CPC A61K 36/3482; A61K 31/658; A61P 1/00; A61P 25/04; A61P 25/22

(57) ABSTRACT

This disclosure relates to non-naturally occurring hemp extracts comprising cannabinoids and related treatment methods.

### CROSS-REFERENCE TO RELATED APPLICATIONS

This application is a Continuation of PCT International Application No. PCT/US2023/064095, filed Mar. 10, 2023, which claims the benefit of U.S. Provisional Application No. 63/269,127, filed Mar. 10, 2022, the entire disclosure of each of which is hereby incorporated by reference.

### FIELD

This disclosure relates to non-naturally occurring or engineered compositions and methods comprising one or more hemp extracts for the treatment of diseases, disorders, syndromes, and/or conditions in animals in need.

### BACKGROUND

Industrial hemp products with 0.3% or less of tetrahydrocannabinol (THC) are legal according to the Industrial Hemp Act. These products alone or when combined with other cannabinoids (CBDs) are reported to have health benefits including analgesic, anti-anxiety, anti-inflammatory, anti-anxiolytic, and anti-epileptic. There are numerous companies selling hemp products including CBD oil claiming that they are safe and effective for various medical conditions in humans and animals. In the absence of an optimal treatment using traditional pharmacological agents for patients in need, other potentially efficacious pharmacological agents such as CBDs are often sought. However, there is very little published data to support safety and efficacy claims for the use of these products in human and veterinary patients.

Thus, there is a need for safer and more targeted hemp products.

### SUMMARY

In one aspect, the present disclosure provides a hemp extract comprising 20 mg/mL to 70 mg/mL of cannabidiol and 20 mg/mL to 70 mg/mL cannabidiolic acid. In some embodiments, other cannabinoids are present at less than 5 mg/mL, individually. In some embodiments, the hemp extract comprises 20 mg/mL to 40 mg/mL of cannabidiol and 20 mg/mL to 40 mg/mL of cannabidiolic acid. In some embodiments, the hemp extract comprises 25 mg/mL to 35 mg/mL of cannabidiol and 25 mg/mL to 35 mg/mL of cannabidiolic acid. In some embodiments, the hemp extract comprises 30 mg/mL to 40 mg/mL of cannabidiol and 30 mg/mL to 40 mg/mL of cannabidiolic acid. In some embodiments, the hemp extract comprises 30 mg/mL to 60 mg/mL of cannabidiol and 30 mg/mL to 60 mg/mL of cannabidiolic acid. In some embodiments, the hemp extract comprises 40 mg/mL to 50 mg/mL of cannabidiol and 40 mg/mL to 50 mg/mL of cannabidiolic acid. In some embodiments, the ratio of cannabidiol to cannabidiolic acid is about 0.6:1 to about 1:0.6.

In another aspect, the present disclosure provides a hemp extract comprising 20 mg/mL to 70 mg/mL of cannabigerol and 25 mg/mL to 70 mg/mL cannabigerolic acid. In some embodiments, other cannabinoids are present at less than 5 mg/mL, individually. In some embodiments, the hemp extract comprises 25 mg/mL to 35 mg/mL of cannabigerol and 30 mg/mL to 40 mg/mL of cannabigerolic acid. In some embodiments, the hemp extract comprises 28 mg/mL to 32 mg/mL of cannabigerol and 32 mg/mL to 37 mg/mL of cannabigerolic acid. In some embodiments, the hemp extract comprises 30 mg/mL to 40 mg/mL of cannabigerol and 30 mg/mL to 40 mg/mL of cannabigerolic acid. In some embodiments, the hemp extract comprises 30 mg/mL to 60 mg/mL of cannabigerol and 30 mg/mL to 60 mg/mL of cannabigerolic acid. In some embodiments, the hemp extract comprises 40 mg/mL to 55 mg/mL of cannabigerol and 40 mg/mL to 55 mg/mL of cannabigerolic acid. In some embodiments, the ratio of cannabigerol to cannabigerolic acid is about 0.6:1 to about 1:0.6.

In another aspect, the present disclosure provides a hemp extract comprising 20 mg/mL to 70 mg/mL of cannabidiolic acid; 20 mg/mL to 70 mg/mL cannabigerolic acid; and 1 mg/mL to 10 mg/mL of cannabigerol. In some embodiments, any other cannabinoids are present at less than 5 mg/mL, individually. In some embodiments, the hemp extract comprises 25 mg/mL to 40 mg/mL of cannabidiolic acid; 25 mg/mL to 40 mg/mL cannabigerolic acid; and 2 mg/mL to 8 mg/mL of cannabigerol. In some embodiments, the hemp extract comprises 30 mg/mL to 34 mg/mL of cannabidiolic acid; 30 mg/mL to 34 mg/mL of cannabigerolic acid; and 2 mg/mL to 7 mg/mL of cannabigerol. In some embodiments, the hemp extract comprises 30 mg/mL to 40 mg/mL of cannabidiolic acid; 30 mg/mL to 40 mg/mL cannabigerolic acid; and 1 mg/mL to 10 mg/mL of cannabigerol. In some embodiments, the hemp extract comprises 30 mg/mL to 60 mg/mL of cannabidiolic acid; 30 mg/mL to 60 mg/mL cannabigerolic acid; and 2 mg/mL to 8 mg/mL of cannabigerol. In some embodiments, the hemp extract comprises 40 mg/mL to 55 mg/mL of cannabidiolic acid; 40 mg/mL to 55 mg/mL cannabigerolic acid; and 2 mg/mL to 8 mg/mL of cannabigerol. In some embodiments, the ratio of cannabidiolic acid to cannabigerolic acid is about 0.6:1 to about 1:0.6.

In another aspect, the present disclosure provides a hemp extract comprising 10 mg/mL to 50 mg/mL of cannabidiol; 5 mg/mL to 35 mg/mL cannabidiolic acid; 5 mg/mL to 35 mg/mL of cannabidivarin; 3 mg/mL to 25 mg/mL cannabidivarinic acid; and 3 mg/mL to 20 mg/mL of cannabigerolic acid. In some embodiments, other cannabinoids are present at less than 5 mg/mL, individually. In some embodiments, the hemp extract comprises 15 mg/mL to 25 mg/mL of cannabidiol; 10 mg/mL to 20 mg/mL cannabidiolic acid; 10 mg/mL to 20 mg/mL of cannabidivarin; 5 mg/mL to 15 mg/mL cannabidivarinic acid; and 5 mg/mL to 10 mg/mL of cannabigerolic acid. In some embodiments, the hemp extract comprises 18 mg/mL to 22 mg/mL of cannabidiol; 13 mg/mL to 17 mg/mL cannabidiolic acid; 13 mg/mL to 17 mg/mL of cannabidivarin; 8 mg/mL to 13 mg/mL cannabidivarinic acid; and 5 mg/mL to 10 mg/mL of cannabigerolic acid.

In another aspect, the present disclosure provides a hemp extract comprising 20 mg/mL to 40 mg/mL of cannabidiol; 10 mg/mL to 30 mg/mL cannabidiolic acid; 15 mg/mL to 30 mg/mL of cannabidivarin; 10 mg/mL to 20 mg/mL cannabidivarinic acid; and 8 mg/mL to 20 mg/mL of cannabigerolic acid. In some embodiments, other cannabinoids are present at less than 5 mg/mL, individually.

In another aspect, the present disclosure provides a hemp extract comprising 5 mg/mL to 30 mg/mL of cannabidiol; 5 mg/mL to 30 mg/mL cannabidiolic acid; 5 mg/mL to 30 mg/mL of cannabidivarin; and 5 mg/mL to 30 mg/mL cannabidivarinic acid. In some embodiments, the hemp extract comprises 10 mg/mL to 25 mg/mL of cannabidiol; 10 mg/mL to 25 mg/mL cannabidiolic acid; 10 mg/mL to 25 mg/mL of cannabidivarin; and 10 mg/mL to 25 mg/mL cannabidivarinic acid. In some embodiments, the hemp extract comprises 12 mg/mL to 22 mg/mL of cannabidiol; 12 mg/mL to 22 mg/mL cannabidiolic acid; 12 mg/mL to 22 mg/mL of cannabidivarin; and 12 mg/mL to 22 mg/mL cannabidivarinic acid. In some embodiments, other cannabinoids are present at less than 5 mg/mL, individually.

In another aspect, the present disclosure provides a hemp extract comprising 20 mg/mL to 60 mg/mL of cannabidiol; 20 mg/mL to 60 mg/mL of cannabidiolic acid; and 5 mg/mL to 30 mg/mL of cannabinol. In some embodiments, other cannabinoids are present at less than 5 mg/mL, individually. In some embodiments, the hemp extract comprises 20 mg/mL to 40 mg/mL of cannabidiol 20 mg/mL to 40 mg/mL of cannabidiolic acid; and 5 mg/mL to 20 mg/mL of cannabinol. In some embodiments, the hemp extract comprises 25 mg/mL to 35 mg/mL of cannabidiol; 25 mg/mL to 35 mg/mL of cannabidiolic acid; and 8 mg/mL to 12 mg/mL of cannabinol. In some embodiments, the hemp extract comprises 30 mg/mL to 55 mg/mL of cannabidiol; 30 mg/mL to 55 mg/mL of cannabidiolic acid; and 10 mg/mL to 20 mg/mL of cannabinol. In some embodiments, the hemp extract comprises 30 mg/mL to 45 mg/mL of cannabidiol; 30 mg/mL to 45 mg/mL of cannabidiolic acid; and 10 mg/mL to 20 mg/mL of cannabinol. In some embodiments, the ratio of cannabidiol to cannabidiolic acid is about 0.6:1 to about 1:0.6.

In another aspect, the present disclosure provides a hemp extract comprising 20 mg/mL to 50 mg/mL of cannabidiol; 5 mg/mL to 25 mg/mL of cannabigerol; and 5 mg/mL to 25 mg/mL of cannabigerolic acid. In some embodiments, other cannabinoids are present at less than 5 mg/mL, individually. In some embodiments, the hemp extract comprises 25 mg/mL to 45 mg/mL of cannabidiol; 10 mg/mL to 20 mg/mL of cannabigerol; and 10 mg/mL to 20 mg/mL of cannabigerolic acid. In some embodiments, the hemp extract comprises 30 mg/mL to 45 mg/mL of cannabidiol 13 mg/mL to 20 mg/mL of cannabigerol; and 13 mg/mL to 20 mg/mL of cannabigerolic acid.

In some embodiments, the total concentration of cannabinoids is from 50 mg/mL to 130 mg/mL. In some embodiments, the hemp extract further comprises α-pinene; β-myrcene; β-pinene; δ-limonene; linalool; β-caryophyllene; α-humulene; guaiol; caryophyllene oxide; and α-bisabolol. In some embodiments, the hemp extract further comprises β-myrcene; δ-limonene; cis-β-ocimene; linalool; β-caryophyllene; α-humulene; trans-nerolidol; guaiol; caryophyllene oxide; and α-bisabolol. In some embodiments, the concentration of Δ9-tetrahydrocannabinol is insufficient to produce a psychotropic effect. In some embodiments, the concentration of Δ9-tetrahydrocannabinol is less than about 5 mg/mL. In some embodiments, the concentration of Δ9-tetrahydrocannabinol is less than about 2 mg/mL. In some embodiments, the concentration of Δ9-tetrahydrocannabinol is less than about 1 mg/mL. In some embodiments, the concentration of Δ9-tetrahydrocannabinol is less than about 0.5 mg/mL. In some embodiments, the hemp extract is formulated in a carrier. In some embodiments, the carrier is selected from the group consisting of hemp seed oil, linseed oil, olive oil, fish oil, salmon oil, coconut oil, catnip oil, sesame oil, MCT oil, lecithin, NF-971P, and grapeseed oil. In some embodiments, the carrier comprises hemp seed oil. In some embodiments, the carrier comprises sesame oil.

In another aspect, the present disclosure provides a hemp extract comprising 5 mg/mL to 70 mg/mL of cannabidiolic acid; 5 mg/mL to 70 mg/mL of cannabigerolic acid; and 5 mg/mL to 70 mg/mL of cannabidivarinic acid. In some embodiments, other cannabinoids are present at less than 5 mg/mL, individually. In some embodiments, the hemp extract comprises 5 mg/mL to 50 mg/mL of cannabidiolic acid; 5 mg/mL to 50 mg/mL of cannabigerolic acid; and 5 mg/mL to 50 mg/mL of cannabidivarinic acid. In some embodiments, the hemp extract comprises 10 mg/mL to 40 mg/mL of cannabidiolic acid; 10 mg/mL to 40 mg/mL of cannabigerolic acid; and 10 mg/mL to 40 mg/mL of cannabidivarinic acid.

In another aspect, the present disclosure provides a dosage form comprising a hemp extract of the present disclosure and at least one of a pharmaceutically acceptable additive, a flavoring agent, a surfactant, and an adjuvant. In some embodiments, the flavoring agent is selected from the group consisting of peppermint oil, mango extract, beef, poultry, and seafood. In some embodiments, the flavoring agent is selected from the group consisting of peanut butter, catnip oil, chicken liver powder, poultry extract, maltodextrin, butter, and bacon. In some embodiments, the dosage form is formulated as a sublingual spray. In some embodiments, the dosage form is formulated as a water, alcohol, polyethylene glycol, or glycerol soluble solution or cream for topical or transdermal application. In some embodiments, the dosage form is formulated as a gel for buccal or mucosal administration. In some embodiments, the dosage form is formulated as a paste for buccal or mucosal administration. In some embodiments, the dosage form is formulated as a gel. In some embodiments, the dosage form is formulated as a tablet. In some embodiments, the dosage form is formulated as a capsule. In some embodiments, the dosage form is formulated as a hard chewable. In some embodiments, the dosage form is formulated as a soft chewable. In some embodiments, the dosage form is formulated for administration using a nebulizer. In some embodiments, the dosage form is formulated for inhalation. In some embodiments, the dosage form is formulated for administration using a pet collar. In some embodiments, the composition is formulated as an edible product for oral administration. In some embodiments, the dosage form is formulated as a chew for oral administration. In some embodiments, the chew is produced using cold extrusion. In some embodiments, the hemp extract or dosage form is packaged to provide one or more doses of hemp extract per package. In some embodiments, the package is resealable. In some embodiments, one dose of hemp extract is a therapeutically effective amount.

In another aspect, the present disclosure provides a method for treating chronic pain in a subject in need thereof, the method comprising administering to the subject a therapeutically effective amount of a hemp extract or dosage form described herein.

In another aspect, the present disclosure provides a method for treating anxiety in a subject in need thereof, the method comprising administering to the subject a therapeutically effective amount of a hemp extract or dosage form described herein.

In another aspect, the present disclosure provides a method for treating inflammatory bowel disease in a subject in need thereof, the method comprising administering to the subject a therapeutically effective amount of a hemp extract or dosage form described herein.

In another aspect, the present disclosure provides a method for treating a sleep disorder in a subject in need thereof, the method comprising administering to the subject a therapeutically effective amount of a hemp extract or dosage form described herein.

In another aspect, the present disclosure provides a method for treating chronic pain, anxiety, inflammatory bowel disease, or a sleep disorder in a subject in need thereof, the method comprising administering to the subject a therapeutically effective amount of a hemp extract or dosage form described herein.

In some embodiments, the hemp extract or dosage form is administered daily. In some embodiments, the hemp extract or dosage form is administered twice daily. In some embodiments, the hemp extract or dosage form is administered orally. In some embodiments, the hemp extract or dosage form is administered at a specific dosage. In some embodiments, the hemp extract or dosage form is administered at a dosage selected from the group consisting of: 5 mg/kg, 7.5 mg/kg, 10 mg/kg, 20 mg/kg, 30 mg/kg, 50 mg/kg, 60 mg/kg, and 100 mg/kg. In some embodiments, the hemp extract or dosage form is administered at a dosage of 5 mg/kg to 60 mg/kg. In some embodiments, the hemp extract or dosage form is administered at a dosage of 10 mg/kg to 100 mg/kg. In some embodiments, the subject is a veterinary subject. In some embodiments, the subject is a human.

### DETAILED DESCRIPTION

It will be appreciated that for clarity, the following disclosure will describe various aspects of embodiments. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element.

As used herein, when “about” is used to refer to a measurable value such as an amount, a temporal duration, and the like, the term “about” is meant to encompass variations of 55% from the specified value, as such variations are appropriate to perform the disclosed methods.

As used in the specification and in the claims, the term “comprising” may include the embodiments “consisting of” and “consisting essentially of” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “treatment” or “treating,” is defined as the application or administration of a therapeutic agent, i.e., a compound provided herein (alone or in combination with another pharmaceutical agent), to a patient or subject, or application or administration of a therapeutic agent to an isolated tissue or cell line from a patient (e.g., for diagnosis or ex vivo applications), with the purpose to cure, heal, alleviate, relieve, alter, remedy, ameliorate, improve or affect the symptoms of a disease, disorder, syndrome, or condition. Such treatments can be specifically tailored or modified, based on knowledge obtained from the field of pharmacology.

In certain embodiments, the compositions described herein treat and/or reduce the severity of a disease, disorder, syndrome, or condition in a subject. For example, the compositions described herein treat and/or improve one or more symptoms of inflammation, anxiety, sleep disorders, pain (e.g., chronic pain, non-chronic pain, neuropathic pain, neurological dysfunction pain, nociceptive pain, post-operation pain), seizure, epilepsy, osteoarthritis, atopy, allergies, diarrhea (e.g., idiopathic diarrhea), skin wounds, gastrointestinal conditions, behavioral issues, obsessive behaviors, inflammatory bowel disease, dermatological conditions (e.g., pruritus, pyoderma), or anxiety

As used herein, the term “prevent” or “prevention” means no disorder or disease development if none had occurred, or no further disorder or disease development if there had already been development of the disorder or disease. Also considered is the ability of one to prevent some or all of the symptoms associated with a disorder, disease, and/or condition.

As used herein, the term “use” includes any one or more of the following embodiments of the invention, respectively: the use in the treatment of a disease, disorder, syndrome, and/or condition for the manufacture of pharmaceutical compositions for use in the treatment of this disease, disorder, syndrome, and/or condition, e.g., in the manufacture and/or preparation of a medicament; methods of use of compounds of the invention in the treatment of this disease, disorder, syndrome, and/or condition; pharmaceutical preparations having compounds of the invention for the treatment of this disease, disorder, syndrome, and/or condition; and compounds of the invention for use in the treatment of this disease, disorder, syndrome, and/or condition; as appropriate and expedient, if not stated otherwise.

As used herein, the term “patient,” “individual,” or “subject” is intended to include organisms, which are capable of suffering from or afflicted with a disease, disorder, syndrome, and/or condition. Examples of subjects include animals. Examples of subjects include mammals. Examples of subjects include dogs, cats, horses, cows, pigs, sheep, goats, mice, rabbits, rats, birds, fishes, non-human primates, amphibians, reptiles, and transgenic non-human animals. In some embodiments, the subject is a human.

When used with respect to methods of treatment/prevention and the use of the compounds and pharmaceutical compositions thereof described herein, a subject “in need thereof” may be a subject who has been diagnosed with or previously treated for the condition to be treated. With respect to prevention, the individual in need thereof may also be a subject who is at risk for a condition (e.g., a family history of the disease, disorder, syndrome, and/or condition, life-style factors indicative of risk for the disease, disorder, syndrome, and/or condition, etc.). Typically, when a step of administering a compound of the invention is disclosed herein, the invention further contemplates a step of identifying a subject in need of the particular treatment to be administered or having the particular condition to be treated.

As used herein, the term “pharmaceutically acceptable” refers to a material, such as a carrier or diluent, which does not abrogate the biological activity or properties of the compound, and is relatively non-toxic, i.e., the material can be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with an amount (e.g., a stoichiometric amount) of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” or “carrier” means a pharmaceutically acceptable material, composition or carrier, such as a liquid or solid (e.g., a solid filler), stabilizer, dispersing agent, suspending agent, diluent, excipient, thickening agent, solvent or encapsulating material, involved in carrying or transporting a compound useful within the invention within or to the patient or subject such that it can perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient or subject. Some examples of materials that can serve as pharmaceutically acceptable carriers include: sugars, such as lactose, glucose, sucrose, and the like; starches, such as corn starch potato starch, and the like; cellulose, and its derivatives, such as sodium carboxymethyl cellulose, ethyl cellulose, cellulose acetate, and the like; powdered tragacanth; malt; gelatin; talc; excipients, such as cocoa butter, coconut butter, suppository waxes, and the like; oils, such as peanut oil, cottonseed oil, safflower oil, sesame oil, olive oil, corn oil, soybean oil, and the like; glycols, such as propylene glycol and the like; polyols, such as glycerin, sorbitol, mannitol, polyethylene glycol, and the like; esters, such as ethyl oleate, ethyl laurate, and the like; agar; buffering agents, such as magnesium hydroxide, aluminum hydroxide, and the like; surface active agents; alginic acid; water, such as tap water, purified water, distilled water, milli-q water, pyrogen-free water, and the like; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial agents, antifungal agents, antioxidant agents, absorption delaying agents, preservative agents, stabilizing agents, and the like that are compatible with the activity of the compound useful within the invention, and are physiologically acceptable to the patient or subject. One or more supplementary active and/or inactive compounds can also be incorporated into the compositions. The “pharmaceutically acceptable carrier” or “carrier” can further include one or more pharmaceutically acceptable salts of the one or more compounds useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

The term “stabilizer,” as used herein, refers to polymers capable of chemically inhibiting or preventing degradation. Stabilizers are added to formulations of compounds to improve chemical and physical stability of the compound.

As used herein, the term “adjuvant” may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, and/or causes of a disease, or any other desired alteration of a biological system. An appropriate therapeutic amount in any individual case may be determined by one of ordinary skill in the art using routine experimentation.

As used herein, the term “weight percent” is meant to refer to the quantity by weight of a compound and/or component in a composition as the quantity by weight of a constituent component of the composition as a percentage of the weight of the total composition. The weight percent can also be calculated by multiplying the mass fraction by 100. The “mass fraction” is the ratio of one substance of a mass m1 to the mass of the total composition mT such that weight percent=(m1/mT)*100. Wherever “%” is used herein, it is assumed to be weight percent unless labeled otherwise.

“Aqueous buffer” refers to a water solution which resists change in hydronium ion and the hydroxide ion concentration (and consequent pH) upon addition of small amounts of acid or base, or upon dilution. Buffer solutions consist of a weak acid and its conjugate base (more common) and/or a weak base and its conjugate acid (less common). The buffer can be prepared by methods well known in the art with the appropriate buffering agents to give the desired pH value. Examples of the suitable buffering agents include hydrochloric acid, lactic acid, acetic acid, citric acid, malic acid, maleic acid, pyruvic acid, succinic acid, tris-hydroxymethylaminomethane, sodium hydroxide, sodium bicarbonate, phosphoric acid, sodium phosphate, and other biologically acceptable buffering agents. Aqueous buffers are readily available commercially and they can be used in preparation of the compositions of this invention without further treatment.

As used herein, the term “hemp extract” refers to a composition of cannabinoids and terpenes that are isolated from a hemp plant. The terms “hemp extract,” “CBD oil,” and “hemp oil” have the same meaning and are used interchangeably herein. The hemp extract can be obtained by any method known in the art. For example, the hemp extract can be obtained by supercritical (or subcritical) CO₂ extraction, which uses carbon dioxide under high pressure and low temperatures to isolate, preserve and maintain the purity of hemp extract. In an embodiment, the hemp extract is obtained from a supercritical CO₂ extraction. For example, supercritical CO₂ extraction may be performed as described in U.S. Pat. No. 8,895,078, which is incorporated herein by reference in its entirety. Alternatively, a solvent such as petroleum ether, ethanol, methanol, butanol, acetone, dry ice, or olive oil can be used, at room temperature (ambient temperature) with stirring, by passive extraction, heated to a temperature above room temperature, or under reflux, as known in the art to provide the hemp extract. In another embodiment, hemp extract from a butanol extraction is employed as starting material for methods disclosed herein.

In some embodiments a hemp extract is used without undergoing decarboxylation. In some embodiments a hemp extract is used after being decarboxylated. In some embodiments hemp extracts are combined to produce a new hemp extract with a desired composition. In some embodiments, extracts undergo further purification procedures to increase the concentration of cannabinoids of interest before combination.

Cannabinoids are compounds isolated from hemp plants. Classes of cannabinoids include for example, without limitation, cannabichromenes, cannabicyclols, cannabidiols, cannabielsoins, cannabigerols, cannabinols, cannabinodiols, cannabitriols, delta-8-tetrahydrocannabinols, and delta-9-tetrahydrocannabinols. Cannabinoid compounds include for example, without limitation, cannabichromene (CBC), cannabichromenic acid (CBCA), cannabichromevarin (CBCV), cannabichromevarinic acid (CBCVA), cannabicyclol (CBL), cannabicyclolic acid (CBLA), cannabicyclovarin (CBLV), cannabidiol (CBD), cannabidiol monomethylether (CBDM), cannabidiolic acid (CBDA), cannabidiorcol (CBD-C1), cannabidivarin (CBDV), cannabidivarinic acid (CBDVA), cannabielsoic acid B (CBEA-B), cannabielsoin (CBE), cannabielsoin acid A (CBEA-A), cannabigerol (CBG), cannabigerol monomethylether (CBGM), cannabigerolic acid (CBGA), cannabigerolic acid monomethylether (CBGAM), cannabigerovarin (CBGV), cannabigerovarinic acid (CBGVA), cannabinodiol (CBND), cannabinodivarin (CBVD), cannabinol (CBN), cannabinol methylether (CBNM), cannabinol-C2 (CBN-C2), cannabinol-C4 (CBN-C4), cannabinolic acid (CBNA), cannabiorcool (CBN-C1), cannabivarin (CBV), 10-Ethoxy-9-hydroxy-delta-6a-tetrahydrocannabinol, 8,9-Dihydroxy-delta-6a-tetrahydrocannabinol, cannabitriol (CBT), cannabitriolvarin (CBTV), delta-8-tetrahy drocannabinol (Δ8-THC), delta-8-tetrahydrocannabinolic acid (Δ8-THCA), delta-9-tetrahydrocannabinol (THC), delta-9-tetrahydrocannabinol-C4 (THC-C4), delta-9-tetrahydrocannabinolic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorcolic acid (THCA-C1), delta-9-tetrahydrocannabivarin (THCV), delta-9-tetrahydrocannabivarinic acid (THCVA), 10-Oxo-delta-6a-tetrahydrocannabinol (OTHC), cannabichromanon (CBCF), cannabifuran (CBF), cannabiglendol, cannabiripsol (CBR), cannabicitran (CBT), ehydrocannabifuran (DCBF), delta-9-cis-tetrahydrocannabinol (cis-THC), and tryhydroxy-delta-9-tetrahydrocannabinol (triOH-THC).

Suitable methods for measuring the cannabinoid and terpene content in the hemp extract are known in the art. In an embodiment, cannabinoid content is determined using liquid chromatography with mass spectrometry detection (LC-MS). In another embodiment, terpene content is determined using gas chromatography with flame ionization detection (GC-FID) analysis of headspace.

As used herein, the term “flavoring agent” refers to an ingredient that is added to a composition to impart a particular flavor, smell, or other organoleptic property. The flavoring agent can be natural or artificial.

As used herein, the term “oil” refers to a nonpolar viscous liquid that is both hydrophobic and lipophilic. Oils may be isolated from animal, vegetable, or petrochemical products.

As used herein, the term “chew” refers to a product or a portion thereof that has rheological and other texture and organoleptic properties which tend to promote chewing upon the article by a target subject. Generally speaking, a chewable matrix will exhibit sufficient ductility that it is at least slightly malleable when bitten by the target subject and sufficient palatability that the target subject is not deterred by its taste from biting it multiple times. By contrast, “chewable” does not mean merely that an article can be chewed by a subject (i.e., it does not mean merely that some portion of the article will fit within a subject's mouth sufficiently to permit engagement of the subject's teeth against the portion).

The “maximal serum concentration level” of a substance, as used herein, refers to the maximal level of the substance found in a plasma sample following a single administration.

As used herein, the term “cold extrusion” refers to a process for producing edible food products comprising several unit operations including adding, mixing, kneading, pressing, shearing, shaping, and forming, all of which are conducted at or near ambient temperature.

As used herein, the term “psychotropic effect” refers to a modification of brain function that results in an alteration of perception, mood, consciousness, and/or behavior.

As used herein, the term “paste” refers to a product or a portion thereof that has rheological and chemical properties which are consistent with containing and delivering pharmaceutical compositions (e.g., a composition comprising a hemp extract) via the mouth. For example, a paste can be applied to the luminal surfaces of the oral cavity (e.g., gums, inner cheeks). Generally speaking, a paste will be sufficiently spreadable that it can be appropriately applied. Additionally, the paste may be flavored and/or exhibit a mouthfeel or texture that promotes consumption by the animal. Thus, in some embodiments, the paste is palatable for the animal to which the paste is applied. In some embodiments, the paste is hydrophobic. In some embodiments, pastes of the present disclosure can be applied to pets by veterinary professionals or by pets' owners.

As used herein, the term “gel” refers to a product or a portion thereof that is an aggregate of fine particles dispersed in a liquid medium, wherein the medium has become viscous enough to behave in some ways like a solid. Additionally, the gel may be flavored and/or exhibit a mouthfeel or texture that promotes consumption by an animal. Thus, in some embodiments, the gel is palatable for the animal to which the gel is applied. In some embodiments, the gel is hydrophilic. In some embodiments, gels of the present disclosure can be applied to pets by veterinary professionals or by pets' owners.

### Overview

The embodiments disclosed herein provide non-naturally occurring or engineered compositions and methods comprising one or more hemp extracts for the treatment of diseases, disorders, syndromes, and/or conditions in animals in need. The diseases, disorders, syndromes, and/or conditions comprise for example, without limitation, inflammation, anxiety, insomnia, pain (e.g., chronic pain, non-chronic pain, neuropathic pain, neurological dysfunction pain, nociceptive pain, post-operation pain), seizure, epilepsy, osteoarthritis, atopy, allergies, diarrhea (e.g., idiopathic diarrhea), skin wounds, gastrointestinal conditions, behavioral issues, obsessive behaviors, inflammatory bowel disease, dermatological conditions (e.g., pruritus, pyoderma), or anxiety. Clinical trials regarding administering and dosing in treatments are disclosed herein.

### Hemp Extracts—Cannabinoid Compositions

In one aspect, provided herein is a hemp extract comprising:
20 mg/mL to 70 mg/mL of cannabidiol; and
20 mg/mL to 70 mg/mL cannabidiolic acid (termed herein a “CBD-CBDA extract”).

In some embodiments, the CBD-CBDA extract comprises other cannabinoids which are present at reduced concentrations. In some embodiments, other cannabinoids are present at less than 12 mg/mL, individually. In some embodiments, other cannabinoids are present at less than 11 mg/mL, individually. In some embodiments, other cannabinoids are present at less than 10 mg/mL, individually. In some embodiments, other cannabinoids are present at less than 9 mg/mL, individually. In some embodiments, other cannabinoids are present at less than 8 mg/mL, individually. In some embodiments, other cannabinoids are present at less than 7 mg/mL, individually. In some embodiments, other cannabinoids are present at less than 6 mg/mL, individually. In some embodiments, other cannabinoids are present at less than 5 mg/mL, individually. In some embodiments, other cannabinoids are present at less than 4 mg/mL, individually. In some embodiments, other cannabinoids are present at less than 3 mg/mL, individually. In some embodiments, other cannabinoids are present at less than 2 mg/mL, individually. In some embodiments, other cannabinoids are present at less than 1 mg/mL, individually.

In some embodiments, the CBD-CBDA extract comprises cannabidiol at a concentration of 20 mg/mL to 40 mg/mL. In some embodiments, the cannabidiol is present at a concentration of 25 mg/mL to 35 mg/mL. In some embodiments, the cannabidiol is present at a concentration of 30 mg/mL to 40 mg/mL. In some embodiments, the cannabidiol is present at a concentration of 30 mg/mL to 60 mg/mL. In some embodiments, the cannabidiol is present at a concentration of 40 mg/mL to 50 mg/mL. In some embodiments, the cannabidiol is present at a concentration of about 25 mg/mL. In some embodiments, the cannabidiol is present at a concentration of about 30 mg/mL. In some embodiments, the cannabidiol is present at a concentration of about 35 mg/mL. In some embodiments, the hemp extract comprises 1% by weight to 7% by weight of cannabidiol. In some embodiments, the hemp extract comprises 2% by weight to 5% by weight of cannabidiol. In some embodiments, the hemp extract comprises 3% to 4% by weight of cannabidiol. In some embodiments, the hemp extract comprises 3% by weight to 6% by weight of cannabidiol. In some embodiments, the hemp extract comprises 4% by weight to 6% by weight cannabidiol.

In some embodiments, the CBD-CBDA extract comprises cannabidiolic acid at a concentration of 20 mg/mL to 40 mg/mL. In some embodiments, the cannabidiolic acid is present at a concentration of 25 mg/mL to 35 mg/mL. In some embodiments, the cannabidiolic acid is present at a concentration of 30 mg/mL to 40 mg/mL. In some embodiments, the cannabidiolic acid is present at a concentration of 30 mg/mL to 60 mg/mL. In some embodiments, the cannabidiolic acid is present at a concentration of 40 mg/mL to 50 mg/mL. In some embodiments, the cannabidiolic acid is present at a concentration of about 25 mg/mL. In some embodiments, the cannabidiolic acid is present at a concentration of about 30 mg/mL. In some embodiments, the cannabidiolic acid is present at a concentration of about 35 mg/mL. In some embodiments, the hemp extract comprises 1% by weight to 7% by weight of cannabidiolic acid. In some embodiments, the hemp extract comprises 2% by weight to 5% by weight of cannabidiolic acid. In some embodiments, the hemp extract comprises 2% by weight to 4% by weight of cannabidiolic acid. In some embodiments, the hemp extract comprises 2% by weight to 7% by weight of cannabidiolic acid. In some embodiments, the hemp extract comprises 4% by weight to 6% by weight of cannabidiolic acid.

In some embodiments, the CBD-CBDA extract comprises 20 mg/mL to 40 mg/mL of cannabidiol and 20 mg/mL to 40 mg/mL of cannabidiolic acid. In some embodiments, the CBD-CBDA extract comprises 25 mg/mL to 35 mg/mL of cannabidiol and 25 mg/mL to 35 mg/mL of cannabidiolic acid. In some embodiments, the CBD-CBDA extract comprises 30 mg/mL to 40 mg/mL of cannabidiol and 30 mg/mL to 40 mg/mL of cannabidiolic acid. In some embodiments, the CBD-CBDA extract comprises 30 mg/mL to 60 mg/mL of cannabidiol and 30 mg/mL to 60 mg/mL of cannabidiolic acid. In some embodiments the CBD-CBDA extract comprises 40 mg/mL to 50 mg/mL of cannabidiol and 40 mg/mL to 50 mg/mL of cannabidiolic acid.

In some embodiments, the CBD-CBDA extract comprises a specific ratio of cannabidiol to cannabidiolic acid. In an embodiment, the ratio of cannabidiol to cannabidiolic acid is selected from the group consisting of about 1:100, about 1:50, about 1:10, and about 1:1. In an embodiment, the ratio of cannabidiol to cannabidiolic acid is about 0.1:1 to about 1:0.1. In another embodiment, the ratio of cannabidiol to cannabidiolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabidiol to cannabidiolic acid is about 0.6:1 to about 1:0.6. In still another embodiment, the ratio of cannabidiol to cannabidiolic acid is about 1:1.

In some embodiments, a CBD extract is combined with a CBDA extract. In some embodiments, the final extract comprises 3% to 9% by weight of each extract, respectively. In some embodiments, the final extract comprises about 6.3% by weight of each extract, respectively.

In another aspect, provided herein is a hemp extract comprising:
20 mg/mL to 70 mg/mL of cannabigerol; and
25 mg/mL to 70 mg/mL cannabigerolic acid, (termed herein a “CBG-CBGA” extract).

In some embodiments, the CBG-CBGA extract comprises other cannabinoids which are present at reduced concentrations. In some embodiments, other cannabinoids are present at less than 5 mg/mL, individually. In some embodiments, other cannabinoids are present at less than 4 mg/mL, individually. In some embodiments, other cannabinoids are present at less than 3 mg/mL, individually. In some embodiments, other cannabinoids are present at less than 2 mg/mL, individually. In some embodiments, other cannabinoids are present at less than 1 mg/mL, individually.

In some embodiments, the CBG-CBGA extract comprises cannabigerol at a concentration of 25 mg/mL to 35 mg/mL. In some embodiments, the cannabigerol is present at a concentration of 28 mg/mL to 32 mg/mL. In some embodiments, the cannabigerol is present at a concentration of 30 mg/mL to 40 mg/mL. In some embodiments, the cannabigerol is present at a concentration of 30 mg/mL to 60 mg/mL. In some embodiments, the cannabigerol is present at a concentration of 40 mg/mL to 55 mg/mL. In some embodiments, the cannabigerol is present at a concentration of about 28 mg/mL. In some embodiments, the cannabigerol is present at a concentration of about 29 mg/mL. In some embodiments, the cannabigerol is present at a concentration of about 30 mg/mL. In some embodiments, the cannabigerol is present at a concentration of about 31 mg/mL. In some embodiments, the cannabigerol is present at a concentration of about 32 mg/mL. In some embodiments, the hemp extract comprises 1% by weight to 7% by weight of cannabigerol. In some embodiments, the hemp extract comprises 1% by weight to 5% by weight of cannabigerol. In some embodiments, the hemp extract comprises 2% by weight to 5% by weight of cannabigerol. In some embodiments, the hemp extract comprises 3% by weight to 7% by weight of cannabigerol. In some embodiments, the hemp extract comprises 3% by weight to 6% by weight of cannabigerol.

In some embodiments, the CBG-CBGA extract comprises cannabigerolic acid at a concentration of 30 mg/mL to 40 mg/mL. In some embodiments, the cannabigerolic acid is present at a concentration of 32 mg/mL to 37 mg/mL. In some embodiments, the cannabigerolic acid is present at a concentration of 30 mg/mL to 40 mg/mL. In some embodiments, the cannabigerolic acid is present at a concentration of 30 mg/mL to 60 mg/mL. In some embodiments, the cannabigerolic acid is present at a concentration of 40 mg/mL to 55 mg/mL. In some embodiments, the cannabigerolic acid is present at a concentration of about 32 mg/mL. In some embodiments, the cannabigerolic acid is present at a concentration of about 33 mg/mL. In some embodiments, the cannabigerolic acid is present at a concentration of about 34 mg/mL. In some embodiments, the cannabigerolic acid is present at a concentration of about 35 mg/mL. In some embodiments, the cannabigerolic acid is present at a concentration of about 36 mg/mL. In some embodiments, the cannabigerolic acid is present at a concentration of about 37 mg/mL. In some embodiments, the hemp extract comprises 1% by weight to 7% by weight of cannabigerolic acid. In some embodiments, the hemp extract comprises 1% by weight to 5% by weight of cannabigerolic acid. In some embodiments, the hemp extract comprises 2% by weight to 5% by weight of cannabigerolic acid. In some embodiments, the hemp extract comprises 3% by weight to 7% by weight of cannabigerolic acid. In some embodiments, the hemp extract comprises 3% by weight to 6% by weight of cannabigerolic acid.

In some embodiments, the CBG-CBGA extract comprises 25 mg/mL to 35 mg/mL of cannabigerol and 30 mg/mL to 40 mg/mL of cannabigerolic acid. In some embodiments, the CBG-CBGA extract comprises 28 mg/mL to 32 mg/mL of cannabigerol and 32 mg/mL to 37 mg/mL of cannabigerolic acid. In some embodiments, the CBG-CBGA extract comprises 30 mg/mL to 40 mg/mL of cannabigerol and 30 mg/mL to 40 mg/mL of cannabigerolic acid. In some embodiments, the CBG-CBGA extract comprises 30 mg/mL to 60 mg/mL of cannabigerol and 30 mg/mL to 60 mg/mL of cannabigerolic acid. In some embodiments, the CBG-CBGA extract comprises 40 mg/mL to 55 mg/mL of cannabigerol and 40 mg/mL to 55 mg/mL of cannabigerolic acid.

In some embodiments, the CBG-CBGA extract comprises a specific ratio of cannabigerol to cannabigerolic acid. In an embodiment, the ratio of cannabigerol to cannabigerolic acid is selected from the group consisting of about 1:100, about 1:50, about 1:10, and about 1:1. In an embodiment, the ratio of cannabigerol to cannabigerolic acid is about 0.1:1 to about 1:0.1. In another embodiment, the ratio of cannabigerol to cannabigerolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabigerol to cannabigerolic acid is about 0.6:1 to about 1:0.6. In still another embodiment, the ratio of cannabigerol to cannabigerolic acid is about 1:1. of any one of claims 1-3, wherein the ratio of cannabigerol to cannabigerolic acid is about 0.6:1 to about 1:0.6.

In some embodiments, a CBG extract is combined with a CBGA extract. In some embodiments, the final extract comprises 3% to 9% by weight of the CBG extract and 2% to 8% by weight of the CBGA. In some embodiments, the final extract comprises about 6.1% by weight of the CBGA extract and about 4.9% by weight of the CBG extract.

In another aspect, provided herein is a hemp extract comprising:
20 mg/mL to 70 mg/mL of cannabidiolic acid;
20 mg/mL to 70 mg/mL cannabigerolic acid; and
1 mg/mL to 10 mg/mL of cannabigerol (termed herein a “CBDA-CBGA-CBG” extract).

In some embodiments, the CBDA-CBGA-CBG extract comprises other cannabinoids which are present at reduced concentrations. In some embodiments, other cannabinoids are present at less than 5 mg/mL, individually. In some embodiments, other cannabinoids are present at less than 4 mg/mL, individually. In some embodiments, other cannabinoids are present at less than 3 mg/mL, individually. In some embodiments, other cannabinoids are present at less than 2 mg/mL, individually. In some embodiments, other cannabinoids are present at less than 1 mg/mL, individually.

In some embodiments, the CBDA-CBGA-CBG extract comprises cannabigerol at a concentration of 2 mg/mL to 8 mg/mL. In some embodiments, the cannabigerol is present at a concentration of 2 mg/mL to 7 mg/mL. In some embodiments, the cannabigerol is present at a concentration of about 2 mg/mL. In some embodiments, the cannabigerol is present at a concentration of about 3 mg/mL. In some embodiments, the cannabigerol is present at a concentration of about 4 mg/mL. In some embodiments, the cannabigerol is present at a concentration of about 5 mg/mL. In some embodiments, the cannabigerol is present at a concentration of about 6 mg/mL. In some embodiments, the cannabigerol is present at a concentration of about 7 mg/mL. In some embodiments, the hemp extract comprises less than 1% by weight of cannabigerol. In some embodiments, the hemp extract comprises less than 0.5% by weight of cannabigerol.

In some embodiments, the CBDA-CBGA-CBG extract comprises cannabigerolic acid at a concentration of 25 mg/mL to 40 mg/mL. In some embodiments, the cannabigerolic acid is present at a concentration of 30 mg/mL to 34 mg/mL. In some embodiments, the cannabigerolic acid is present at a concentration of 30 mg/mL to 40 mg/mL. In some embodiments, the cannabigerolic acid is present at a concentration of 30 mg/mL to 60 mg/mL. In some embodiments, the cannabigerolic acid is present at a concentration of 40 mg/mL to 55 mg/mL. In some embodiments, the cannabigerolic acid is present at a concentration of about 30 mg/mL. In some embodiments, the cannabigerolic acid is present at a concentration of about 31 mg/mL. In some embodiments, the cannabigerolic acid is present at a concentration of about 32 mg/mL. In some embodiments, the cannabigerolic acid is present at a concentration of about 33 mg/mL. In some embodiments, the cannabigerolic acid is present at a concentration of about 34 mg/mL. In some embodiments, the cannabigerolic acid is present at a concentration of about 35 mg/mL. In some embodiments, the hemp extract comprises 1% by weight to 7% by weight of cannabigerolic acid. In some embodiments, the hemp extract comprises 2% by weight to 5% by weight of cannabigerolic acid. In some embodiments, the hemp extract comprises 2% by weight to 5% by weight of cannabigerolic acid. In some embodiments, the hemp extract comprises 3% by weight to 6% by weight of cannabigerolic acid.

In some embodiments, the CBDA-CBGA-CBG extract comprises cannabidiolic acid at a concentration of 25 mg/mL to 40 mg/mL. In some embodiments, the cannabidiolic acid is present at a concentration of 30 mg/mL to 34 mg/mL. In some embodiments, the cannabidiolic acid is present at a concentration of 30 mg/mL to 40 mg/mL. In some embodiments, the cannabidiolic acid is present at a concentration of 30 mg/mL to 60 mg/mL. In some embodiments, the cannabidiolic acid is present at a concentration of 40 mg/mL to 55 mg/mL. In some embodiments, the cannabidiolic acid is present at a concentration of about 30 mg/mL. In some embodiments, the cannabidiolic acid is present at a concentration of about 31 mg/mL. In some embodiments, the cannabidiolic acid is present at a concentration of about 32 mg/mL. In some embodiments, the cannabidiolic acid is present at a concentration of about 33 mg/mL. In some embodiments, the cannabidiolic acid is present at a concentration of about 34 mg/mL. In some embodiments, the cannabidiolic acid is present at a concentration of about 35 mg/mL. In some embodiments, the hemp extract comprises 1% by weight to 7% by weight of cannabidiolic acid. In some embodiments, the hemp extract comprises 2% by weight to 5% by weight of cannabidiolic acid. In some embodiments, the hemp extract comprises 2% by weight to 5% by weight of cannabidiolic acid. In some embodiments, the hemp extract comprises 3% by weight to 6% by weight of cannabidiolic acid.

In some embodiments, the CBDA-CBGA-CBG extract comprises 25 mg/mL to 40 mg/mL of cannabidiolic acid; 25 mg/mL to 40 mg/mL cannabigerolic acid; and 2 mg/mL to 8 mg/mL of cannabigerol. In some embodiments, the CBDA-CBGA-CBG extract comprises 30 mg/mL to 34 mg/mL of cannabidiolic acid; 30 mg/mL to 34 mg/mL of cannabigerolic acid; and 2 mg/mL to 7 mg/mL of cannabigerol. In some embodiments, the CBDA-CBGA-CBG extract comprises 30 mg/mL to 40 mg/mL of cannabidiolic acid; 30 mg/mL to 40 mg/mL cannabigerolic acid; and 1 mg/mL to 10 mg/mL of cannabigerol. In some embodiments, the CBDA-CBGA-CBG extract comprises 30 mg/mL to 60 mg/mL of cannabidiolic acid; 30 mg/mL to 60 mg/mL of cannabigerolic acid; and 2 mg/mL to 8 mg/mL of cannabigerol. In some embodiments, the CBDA-CBGA-CBG extract comprises 40 mg/mL to 55 mg/mL of cannabidiolic acid; 40 mg/mL to 55 mg/mL of cannabigerolic acid; and 2 mg/mL to 8 mg/mL of cannabigerol.

In some embodiments, the CBDA-CBGA-CBG extract comprises a specific ratio of cannabidiolic acid to cannabigerolic acid. In an embodiment, the ratio of cannabidiolic acid to cannabigerolic acid is selected from the group consisting of about 1:100, about 1:50, about 1:10, and about 1:1. In an embodiment, the ratio of cannabidiolic acid to cannabigerolic acid is about 0.1:1 to about 1:0.1. In another embodiment, the ratio of cannabidiolic acid to cannabigerolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabidiolic acid to cannabigerolic acid is about 0.6:1 to about 1:0.6. In still another embodiment, the ratio of cannabidiolic acid to cannabigerolic acid is about 1:1. of any one of claims 1-3, wherein the ratio of cannabidiolic acid to cannabigerolic acid is about 0.6:1 to about 1:0.6.

In some embodiments, a CBDA extract is combined with a CBGA extract. In some embodiments, the final extract comprises 3% to 9% by weight of the CBDA extract and 3% to 8% by weight of the CBGA. In some embodiments, the final extract comprises about 5.7% by weight of the CBGA extract and about 6.4% by weight of the CBDA extract.

In another aspect, provided herein is a hemp extract comprising:
10 mg/mL to 50 mg/mL of cannabidiol;
5 mg/mL to 35 mg/mL cannabidiolic acid;
5 mg/mL to 35 mg/mL of cannabidivarin;
3 mg/mL to 25 mg/mL cannabidivarinic acid; and
3 mg/mL to 20 mg/mL of cannabigerolic acid (termed herein a “CBD-CBDA-CBDV-CBDVA-CBGA” extract).

In some embodiments, the CBD-CBDA-CBDV-CBDVA-CBGA extract comprises other cannabinoids which are present at reduced concentrations. In some embodiments, other cannabinoids are present at less than 7 mg/mL, individually. In some embodiments, other cannabinoids are present at less than 6 mg/mL, individually. In some embodiments, other cannabinoids are present at less than 5 mg/mL, individually. In some embodiments, other cannabinoids are present at less than 4 mg/mL, individually. In some embodiments, other cannabinoids are present at less than 3 mg/mL, individually. In some embodiments, other cannabinoids are present at less than 2 mg/mL, individually. In some embodiments, other cannabinoids are present at less than 1 mg/mL, individually.

In some embodiments, the CBD-CBDA-CBDV-CBDVA-CBGA extract comprises cannabidivarin at a concentration of 10 mg/mL to 20 mg/mL. In some embodiments, the extract comprises cannabidivarin at a concentration of 13 mg/mL to 17 mg/mL. In some embodiments, the extract comprises cannabidivarin at a concentration of 15 mg/mL to 30 mg/mL. In some embodiments, the cannabidivarin is present at a concentration of about 13 mg/mL. In some embodiments, the cannabidivarin is present at a concentration of about 14 mg/mL. In some embodiments, the cannabidivarin is present at a concentration of about 15 mg/mL. In some embodiments, the cannabidivarin is present at a concentration of about 16 mg/mL. In some embodiments, the cannabidivarin is present at a concentration of about 17 mg/mL. In some embodiments, the cannabidivarin is present at a concentration of about 18 mg/mL. In some embodiments, the hemp extract comprises 0.5% by weight to 5% by weight of cannabidivarin. In some embodiments, the hemp extract comprises 0.5% by weight to 2% by weight of cannabidivarin. In some embodiments, the hemp extract comprises 1% by weight to 3% by weight of cannabidivarin.

In some embodiments, the CBD-CBDA-CBDV-CBDVA-CBGA extract comprises cannabidivarinic acid at a concentration of 5 mg/mL to 15 mg/mL. In some embodiments, the extract comprises cannabidivarinic acid at a concentration of 8 mg/mL to 13 mg/mL. In some embodiments, the extract comprises cannabidivarinic acid at a concentration of 10 mg/mL to 20 mg/mL. In some embodiments, the cannabidivarinic acid is present at a concentration of about 8 mg/mL. In some embodiments, the cannabidivarinic acid is present at a concentration of about 9 mg/mL. In some embodiments, the cannabidivarinic acid is present at a concentration of about 10 mg/mL. In some embodiments, the cannabidivarinic acid is present at a concentration of about 11 mg/mL. In some embodiments, the cannabidivarinic acid is present at a concentration of about 12 mg/mL. In some embodiments, the cannabidivarinic acid is present at a concentration of about 13 mg/mL. In some embodiments, the hemp extract comprises 0.5% by weight to 4% by weight of cannabidivarinic acid. In some embodiments, the hemp extract comprises 0.5% by weight to 2% by weight of cannabidivarinic acid. In some embodiments, the hemp extract comprises 1% by weight to 3% by weight of cannabidivarinic acid.

In some embodiments, the CBD-CBDA-CBDV-CBDVA-CBGA extract comprises cannabidiol at a concentration of 15 mg/mL to 25 mg/mL. In some embodiments, the cannabidiol is present at a concentration of 18 mg/mL to 22 mg/mL. In some embodiments, the cannabidiol is present at a concentration of 20 mg/mL to 40 mg/mL. In some embodiments, the cannabidiol is present at a concentration of about 18 mg/mL. In some embodiments, the cannabidiol is present at a concentration of about 19 mg/mL. In some embodiments, the cannabidiol is present at a concentration of about 20 mg/mL. In some embodiments, the cannabidiol is present at a concentration of about 21 mg/mL. In some embodiments, the cannabidiol is present at a concentration of about 22 mg/mL. In some embodiments, the hemp extract comprises 1% by weight to 4% by weight of cannabidiol. In some embodiments, the hemp extract comprises 1% by weight to 3% by weight of cannabidiol. In some embodiments, the hemp extract comprises 2% by weight to 4% by weight of cannabidiol.

In some embodiments, the CBD-CBDA-CBDV-CBDVA-CBGA extract comprises cannabigerolic acid at a concentration of 5 mg/mL to 10 mg/mL. In some embodiments, the cannabigerolic acid is present at a concentration of 8 mg/mL to 20 mg/mL. In some embodiments, the cannabigerolic acid is present at a concentration of about 5 mg/mL. In some embodiments, the cannabigerolic acid is present at a concentration of about 6 mg/mL. In some embodiments, the cannabigerolic acid is present at a concentration of about 7 mg/mL. In some embodiments, the cannabigerolic acid is present at a concentration of about 8 mg/mL. In some embodiments, the cannabigerolic acid is present at a concentration of about 9 mg/mL. In some embodiments, the cannabigerolic acid is present at a concentration of about 10 mg/mL. In some embodiments, the hemp extract comprises 0.5% by weight to 4% by weight of cannabigerolic acid. In some embodiments, the hemp extract comprises 0.5% by weight to 3% by weight of cannabigerolic acid. In some embodiments, the hemp extract comprises 1% by weight to 4% by weight of cannabigerolic acid.

In some embodiments, the CBD-CBDA-CBDV-CBDVA-CBGA extract comprises cannabidiolic acid at a concentration of 10 mg/mL to 20 mg/mL. In some embodiments, the cannabidiolic acid is present at a concentration of 13 mg/mL to 17 mg/mL. In some embodiments, the cannabidiolic acid is present at a concentration of about 13 mg/mL. In some embodiments, the cannabidiolic acid is present at a concentration of about 14 mg/mL. In some embodiments, the cannabidiolic acid is present at a concentration of about 15 mg/mL. In some embodiments, the cannabidiolic acid is present at a concentration of about 16 mg/mL. In some embodiments, the cannabidiolic acid is present at a concentration of about 17 mg/mL. In some embodiments, the hemp extract comprises 0.5% by weight to 5% by weight of cannabidiolic acid. In some embodiments, the hemp extract comprises 0.5% by weight to 3% by weight of cannabidiolic acid. In some embodiments, the hemp extract comprises 1% by weight to 4% by weight of cannabidiolic acid.

In some embodiments, the CBD-CBDA-CBDV-CBDVA-CBGA extract comprises 15 mg/mL to 25 mg/mL of cannabidiol; 10 mg/mL to 20 mg/mL cannabidiolic acid; 10 mg/mL to 20 mg/mL of cannabidivarin; 5 mg/mL to 15 mg/mL cannabidivarinic acid; and 5 mg/mL to 10 mg/mL of cannabigerolic acid. In some embodiments, the CBD-CBDA-CBDV-CBDVA-CBGA extract comprises 18 mg/mL to 22 mg/mL of cannabidiol; 13 mg/mL to 17 mg/mL cannabidiolic acid; 13 mg/mL to 17 mg/mL of cannabidivarin; 8 mg/mL to 13 mg/mL cannabidivarinic acid; and 5 mg/mL to 10 mg/mL of cannabigerolic acid.

In some embodiments, a CBDVA extract is combined with a CBDV extract. In some embodiments, the final extract comprises 3% to 9% by weight of the CBDV extract and 3% to 9% by weight of the CBDVA. In some embodiments, the final extract comprises about 6.15% by weight of the CBDV extract and about 6.15% by weight of the CBDVA extract. Other cannabinoids present in the CBDVA and/or CBDV extracts are the source of the remaining cannabinoids.

In another aspect, provided herein is a hemp extract comprising:
5 mg/mL to 30 mg/mL of cannabidiol;
5 mg/mL to 30 mg/mL cannabidiolic acid;
5 mg/mL to 30 mg/mL of cannabidivarin; and
5 mg/mL to 30 mg/mL cannabidivarinic acid (termed herein a “CBD-CBDA-CBDV-CBDVA” extract).

In some embodiments, the CBD-CBDA-CBDV-CBDVA extract comprises other cannabinoids which are present at reduced concentrations. In some embodiments, other cannabinoids are present at less than 7 mg/mL, individually. In some embodiments, other cannabinoids are present at less than 6 mg/mL, individually. In some embodiments, other cannabinoids are present at less than 5 mg/mL, individually. In some embodiments, other cannabinoids are present at less than 4 mg/mL, individually. In some embodiments, other cannabinoids are present at less than 3 mg/mL, individually. In some embodiments, other cannabinoids are present at less than 2 mg/mL, individually. In some embodiments, other cannabinoids are present at less than 1 mg/mL, individually.

In some embodiments, the CBD-CBDA-CBDV-CBDVA extract comprises cannabidivarin at a concentration of 10 mg/mL to 25 mg/mL. In some embodiments, the extract comprises cannabidivarin at a concentration of 12 mg/mL to 22 mg/mL. In some embodiments, the cannabidivarin is present at a concentration of about 17 mg/mL. In some embodiments, the cannabidivarin is present at a concentration of about 18 mg/mL.

In some embodiments, the CBD-CBDA-CBDV-CBDVA extract comprises cannabidivarinic acid at a concentration of 10 mg/mL to 25 mg/mL. In some embodiments, the extract comprises cannabidivarinic acid at a concentration of 12 mg/mL to 22 mg/mL. In some embodiments, the cannabidivarinic acid is present at a concentration of about 17 mg/mL. In some embodiments, the cannabidivarinic acid is present at a concentration of about 18 mg/mL.

In some embodiments, the CBD-CBDA-CBDV-CBDVA extract comprises cannabidiol at a concentration of 10 mg/mL to 25 mg/mL. In some embodiments, the cannabidiol is present at a concentration of 12 mg/mL to 22 mg/mL. In some embodiments, the cannabidiol is present at a concentration of about 17 mg/mL. In some embodiments, the cannabidiol is present at a concentration of about 18 mg/mL.

In some embodiments, the CBD-CBDA-CBDV-CBDVA extract comprises cannabidiolic acid at a concentration of 10 mg/mL to 25 mg/mL. In some embodiments, the cannabidiolic acid is present at a concentration of 12 mg/mL to 22 mg/mL. In some embodiments, the cannabidiolic acid is present at a concentration of about 17 mg/mL. In some embodiments, the cannabidiolic acid is present at a concentration of about 18 mg/mL.

In some embodiments, the CBD-CBDA-CBDV-CBDVA-CBGA extract comprises 10 mg/mL to 25 mg/mL of cannabidiol; 10 mg/mL to 25 mg/mL cannabidiolic acid; 10 mg/mL to 25 mg/mL of cannabidivarin; and 10 mg/mL to 25 mg/mL cannabidivarinic acid. In some embodiments, the CBD-CBDA-CBDV-CBDVA-CBGA extract comprises 12 mg/mL to 22 mg/mL of cannabidiol; 12 mg/mL to 22 mg/mL cannabidiolic acid; 12 mg/mL to 22 mg/mL of cannabidivarin; and 12 mg/mL to 22 mg/mL cannabidivarinic acid.

In another aspect, provide herein is a hemp extract comprising:
20 mg/mL to 60 mg/mL of cannabidiol;
20 mg/mL to 60 mg/mL of cannabidiolic acid; and
5 mg/mL to 30 mg/mL of cannabinol (termed herein a “CBD-CBDA-CBN” extract).

In some embodiments, the CBD-CBDA-CBN extract comprises other cannabinoids which are present at reduced concentrations. In some embodiments, other cannabinoids are present at less than 5 mg/mL, individually. In some embodiments, other cannabinoids are present at less than 4 mg/mL, individually. In some embodiments, other cannabinoids are present at less than 3 mg/mL, individually. In some embodiments, other cannabinoids are present at less than 2 mg/mL, individually. In some embodiments, other cannabinoids are present at less than 1 mg/mL, individually.

In some embodiments, the CBD-CBDA-CBN extract comprises cannabidiolic acid at a concentration of 20 mg/mL to 40 mg/mL. In some embodiments, the cannabidiolic acid is present at a concentration of 25 mg/mL to 35 mg/mL. In some embodiments, the cannabidiolic acid is present at a concentration of 30 mg/mL to 55 mg/mL. In some embodiments, the cannabidiolic acid is present at a concentration of 30 mg/mL to 45 mg/mL. In some embodiments, the cannabidiolic acid is present at a concentration of about 25 mg/mL. In some embodiments, the cannabidiolic acid is present at a concentration of about 26 mg/mL. In some embodiments, the cannabidiolic acid is present at a concentration of about 27 mg/mL. In some embodiments, the cannabidiolic acid is present at a concentration of about 28 mg/mL. In some embodiments, the cannabidiolic acid is present at a concentration of about 29 mg/mL. In some embodiments, the cannabidiolic acid is present at a concentration of about 30 mg/mL. In some embodiments, the cannabidiolic acid is present at a concentration of about 40 mg/mL. In some embodiments, the hemp extract comprises 1% by weight to 7% by weight of cannabidiolic acid. In some embodiments, the hemp extract comprises 1% by weight to 4% by weight of cannabidiolic acid. In some embodiments, the hemp extract comprises 2% by weight to 5% by weight of cannabidiolic acid.

In some embodiments, the CBD-CBDA-CBN extract comprises cannabinol at a concentration of 8 mg/mL to 12 mg/mL. In some embodiments, the cannabinol is present at a concentration of 5 mg/mL to 20 mg/mL. In some embodiments, the cannabinol is present at a concentration of 10 mg/mL to 20 mg/mL. In some embodiments, the cannabinol is present at a concentration of about 8 mg/mL. In some embodiments, the cannabinol is present at a concentration of about 9 mg/mL. In some embodiments, the cannabinol is present at a concentration of about 10 mg/mL. In some embodiments, the cannabinol is present at a concentration of about 11 mg/mL. In some embodiments, the cannabinol is present at a concentration of about 12 mg/mL. In some embodiments, the cannabinol is present at a concentration of about 13 mg/mL. In some embodiments, the cannabinol is present at a concentration of about 14 mg/mL. In some embodiments, the cannabinol is present at a concentration of about 15 mg/mL. In some embodiments, the hemp extract comprises 0.5% by weight to 4% by weight of cannabinol. In some embodiments, the hemp extract comprises 0.5% by weight to 3% by weight of cannabinol. In some embodiments, the hemp extract comprises 1% by weight to 3% by weight of cannabinol. In some embodiments, the hemp extract comprises 1% by weight to 2% by weight of cannabinol.

In some embodiments, the CBD-CBDA-CBN extract comprises cannabidiol at a concentration of 20 mg/mL to 40 mg/mL. In some embodiments, the cannabidiol is present at a concentration of 25 mg/mL to 35 mg/mL. In some embodiments, the cannabidiol is present at a concentration of 30 mg/mL to 55 mg/mL. In some embodiments, the cannabidiol is present at a concentration of 30 mg/mL to 45 mg/mL. In some embodiments, the cannabidiol is present at a concentration of about 25 mg/mL. In some embodiments, the cannabidiol is present at a concentration of about 26 mg/mL. In some embodiments, the cannabidiol is present at a concentration of about 27 mg/mL. In some embodiments, the cannabidiol is present at a concentration of about 28 mg/mL. In some embodiments, the cannabidiol is present at a concentration of about 29 mg/mL. In some embodiments, the cannabidiol is present at a concentration of about 30 mg/mL. In some embodiments, the cannabidiol is present at a concentration of about 36 mg/mL. In some embodiments, the cannabidiol is present at a concentration of about 37 mg/mL. In some embodiments, the cannabidiol is present at a concentration of about 38 mg/mL. In some embodiments, the hemp extract comprises 1% by weight to 6% by weight of cannabidiol. In some embodiments, the hemp extract comprises 1.5% by weight to 4% by weight of cannabidiol. In some embodiments, the hemp extract comprises 2.5% by weight to 5% by weight of cannabidiol.

In some embodiments, the CBD-CBDA-CBN extract comprises 20 mg/mL to 40 mg/mL of cannabidiol; 20 mg/mL to 40 mg/mL of cannabidiolic acid; and 8 mg/mL to 12 mg/mL of cannabinol. In some embodiments, the CBD-CBDA-CBN extract comprises 25 mg/mL to 35 mg/mL of cannabidiol; 25 mg/mL to 35 mg/mL of cannabidiolic acid; and 8 mg/mL to 12 mg/mL of cannabinol. In some embodiments, the CBD-CBDA-CBN extract comprises 30 mg/mL to 55 mg/mL of cannabidiol; 30 mg/mL to 55 mg/mL of cannabidiolic acid; and 10 mg/mL to 20 mg/mL of cannabinol. In some embodiments, the CBD-CBDA-CBN extract comprises 30 mg/mL to 45 mg/mL of cannabidiol; 30 mg/mL to 45 mg/mL of cannabidiolic acid; and 10 mg/mL to 20 mg/mL of cannabinol.

In some embodiments, the CBD-CBDA-CBN extract comprises a specific ratio of cannabidiol to cannabidiolic acid. In an embodiment, the ratio of cannabidiolic acid to cannabigerolic acid is selected from the group consisting of about 1:100, about 1:50, about 1:10, and about 1:1. In an embodiment, the ratio of cannabidiolic acid to cannabigerolic acid is about 0.1:1 to about 1:0.1. In another embodiment, the ratio of cannabidiolic acid to cannabigerolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabidiolic acid to cannabigerolic acid is about 0.6:1 to about 1:0.6. In still another embodiment, the ratio of cannabidiolic acid to cannabigerolic acid is about 1:1. of any one of claims 1-3, wherein the ratio of cannabidiolic acid to cannabigerolic acid is about 0.6:1 to about 1:0.6.

In some embodiments, a CBDA extract, CBD extract, and a CBN extract are combined. In some embodiments, the final extract comprises 3% to 8% by weight of the CBDA extract, 3% to 8% by weight of the CBD extract, and 0.5% to 3% by weight of the CBN extract. In some embodiments, the final extract comprises about 5.4% by weight of the CBDA extract, about 5.35% by weight of the CBD extract, and about 1.11% by weight of the CBN extract.

In another aspect, provided herein is a hemp extract comprising:
20 mg/mL to 50 mg/mL of cannabidiol;
5 mg/mL to 25 mg/mL of cannabigerol; and
5 mg/mL to 25 mg/mL of cannabigerolic acid (termed herein a “CBD-CBG-CBGA” extract).

In some embodiments, the CBD-CBG-CBGA extract comprises other cannabinoids which are present at reduced concentrations. In some embodiments, other cannabinoids are present at less than 5 mg/mL, individually. In some embodiments, other cannabinoids are present at less than 4 mg/mL, individually. In some embodiments, other cannabinoids are present at less than 3 mg/mL, individually. In some embodiments, other cannabinoids are present at less than 2 mg/mL, individually. In some embodiments, other cannabinoids are present at less than 1 mg/mL, individually.

In some embodiments, the CBD-CBG-CBGA extract comprises cannabidiol at a concentration of 25 mg/mL to 45 mg/mL. In some embodiments, the cannabidiol is present at a concentration of 30 mg/mL to 45 mg/mL. In some embodiments, the cannabidiol is present at a concentration of about 41 mg/mL. In some embodiments, the cannabidiol is present at a concentration of about 40 mg/mL. In some embodiments, the cannabidiol is present at a concentration of about 39 mg/mL. In some embodiments, the cannabidiol is present at a concentration of about 36 mg/mL. In some embodiments, the cannabidiol is present at a concentration of about 37 mg/mL. In some embodiments, the cannabidiol is present at a concentration of about 38 mg/mL.

In some embodiments, the CBD-CBG-CBGA extract comprises cannabigerol at a concentration of 10 mg/mL to 20 mg/mL. In some embodiments, the cannabigerol is present at a concentration of 13 mg/mL to 20 mg/mL. In some embodiments, the cannabigerol is present at a concentration of about 14 mg/mL. In some embodiments, the cannabigerol is present at a concentration of about 15 mg/mL. In some embodiments, the cannabigerol is present at a concentration of about 16 mg/mL. In some embodiments, the cannabigerol is present at a concentration of about 17 mg/mL. In some embodiments, the cannabigerol is present at a concentration of about 18 mg/mL.

In some embodiments, the CBD-CBG-CBGA extract comprises cannabigerolic acid at a concentration of 10 mg/mL to 20 mg/mL. In some embodiments, the cannabigerolic acid is present at a concentration of 10 mg/mL to 20 mg/mL. In some embodiments, the cannabigerolic acid is present at a concentration of 13 mg/mL to 20 mg/mL. In some embodiments, the cannabigerolic acid is present at a concentration of about 14 mg/mL. In some embodiments, the cannabigerolic acid is present at a concentration of about 15 mg/mL. In some embodiments, the cannabigerolic acid is present at a concentration of about 16 mg/mL. In some embodiments, the cannabigerolic acid is present at a concentration of about 17 mg/mL. In some embodiments, the cannabigerolic acid is present at a concentration of about 18 mg/mL.

In some embodiments, the CBD-CBG-CBGA extract comprises 25 mg/mL to 45 mg/mL of cannabidiol; 10 mg/mL to 20 mg/mL of cannabigerol; and 10 mg/mL to 20 mg/mL of cannabigerolic acid. In some embodiments, the CBD-CBG-CBGA extract comprises 30 mg/mL to 45 mg/mL of cannabidiol; 13 mg/mL to 20 mg/mL of cannabigerol; and 13 mg/mL to 20 mg/mL of cannabigerolic acid.

In some embodiments, a CBD extract, CBG extract, and a CBGA extract are combined. In some embodiments, the final extract comprises 2% to 8% by weight of the CBD extract, 0.5% to 3% by weight of the CBG extract, and 0.5% to 3% by weight of the CBGA extract. In some embodiments, the final extract comprises about 4.48% by weight of the CBD extract, about 1.88% by weight of the CBG extract, and about 1.88% by weight of the CBGA extract.

In another aspect, provided herein is a hemp extract comprising:
5 mg/mL to 70 mg/mL cannabidiolic acid;
5 mg/mL to 70 mg/mL of cannabigerolic acid; and
5 mg/mL to 70 mg/mL cannabidivarinic acid (termed herein a “CBDA-CBGA-CBDVA” extract).

In some embodiments, the CBDA-CBGA-CBDVA extract comprises other cannabinoids which are present at reduced concentrations. In some embodiments, other cannabinoids are present at less than 7 mg/mL, individually. In some embodiments, other cannabinoids are present at less than 6 mg/mL, individually. In some embodiments, other cannabinoids are present at less than 5 mg/mL, individually. In some embodiments, other cannabinoids are present at less than 4 mg/mL, individually. In some embodiments, other cannabinoids are present at less than 3 mg/mL, individually. In some embodiments, other cannabinoids are present at less than 2 mg/mL, individually. In some embodiments, other cannabinoids are present at less than 1 mg/mL, individually.

In some embodiments, the CBDA-CBGA-CBDVA extract comprises cannabidivarinic acid at a concentration of 5 mg/mL to 50 mg/mL. In some embodiments, the extract comprises cannabidivarinic acid at a concentration of 10 mg/mL to 40 mg/mL.

In some embodiments, the CBDA-CBGA-CBDVA extract comprises cannabigerolic acid at a concentration of 5 mg/mL to 50 mg/mL. In some embodiments, the cannabigerolic acid is present at a concentration of 10 mg/mL to 40 mg/mL.

In some embodiments, the CBDA-CBGA-CBDVA extract comprises cannabidiolic acid at a concentration of 5 mg/mL to 50 mg/mL. In some embodiments, the cannabidiolic acid is present at a concentration of 10 mg/mL to 40 mg/mL.

In some embodiments, the CBDA-CBGA-CBDVA extract comprises 5 mg/mL to 50 mg/mL of cannabidiolic acid; 5 mg/mL to 50 mg/mL cannabigerolic acid; and 5 mg/mL to 50 mg/mL of cannabidivarinic acid.

### Hemp Extracts—General Characteristics

Any hemp extracts disclosed herein can comprise these characteristics.

In an embodiment, the hemp extract comprises about 0.01 mg/mL of cannabinoids. In an embodiment, the hemp extract comprises about 0.05 mg/mL of cannabinoids. In an embodiment, the hemp extract comprises about 0.1 mg/mL of cannabinoids. In an embodiment, the hemp extract comprises about 0.5 mg/mL of cannabinoids. In an embodiment, the hemp extract comprises about 1 mg/mL of cannabinoids. In an embodiment, the hemp extract comprises about 2 mg/mL of cannabinoids. In an embodiment, the hemp extract comprises about 3 mg/mL of cannabinoids. In an embodiment, the hemp extract comprises about 4 mg/mL of cannabinoids. In an embodiment, the hemp extract comprises about 5 mg/mL of cannabinoids. In an embodiment, the hemp extract comprises about 10 mg/mL of cannabinoids. In an embodiment, the hemp extract comprises about 20 mg/mL of cannabinoids. In an embodiment, the hemp extract comprises about 30 mg/mL of cannabinoids. In an embodiment, the hemp extract comprises about 40 mg/mL of cannabinoids. In an embodiment, the hemp extract comprises about 50 mg/mL of cannabinoids. In an embodiment, the hemp extract comprises about 60 mg/mL of cannabinoids. In an embodiment, the hemp extract comprises about 70 mg/mL of cannabinoids. In an embodiment, the hemp extract comprises about 80 mg/mL of cannabinoids. In an embodiment, the hemp extract comprises about 90 mg/mL of cannabinoids. In an embodiment, the hemp extract comprises about 100 mg/mL of cannabinoids. In an embodiment, the hemp extract comprises about 110 mg/mL of cannabinoids. In an embodiment, the hemp extract comprises about 120 mg/mL of cannabinoids. In an embodiment, the hemp extract comprises about 130 mg/mL of cannabinoids. In an embodiment, the hemp extract comprises from 50 mg/mL to 130 mg/mL of cannabinoids. In an embodiment, the hemp extract comprises from 60 mg/mL to 120 mg/mL of cannabinoids. In an embodiment, the hemp extract comprises from 70 mg/mL to 120 mg/mL of cannabinoids. In an embodiment, the hemp extract comprises from 50 mg/mL to 80 mg/mL of cannabinoids. In an embodiment, the hemp extract comprises from 80 mg/mL to 120 mg/mL of cannabinoids. In an embodiment, the hemp extract comprises from 85 mg/mL to 115 mg/mL of cannabinoids.

In an embodiment, the hemp extract comprises from 4% w/w to 20% w/w of total cannabinoids. In an embodiment, the hemp extract comprises from 4% w/w to 15% w/w of total cannabinoids. In an embodiment, the hemp extract comprises from 6% w/w to 20% w/w of total cannabinoids. In an embodiment, the hemp extract comprises from 6% w/w to 15% w/w of total cannabinoids. In an embodiment, the hemp extract comprises from 6% w/w to 10% w/w of total cannabinoids. In an embodiment, the hemp extract comprises from 8% w/w to 20% w/w of total cannabinoids. In an embodiment, the hemp extract comprises from 8% w/w to 15% w/w of total cannabinoids. In an embodiment, the hemp extract comprises from 9% w/w to 13% w/w of total cannabinoids.

In an embodiment, provided herein is a hemp extract comprising one or more of.
α-pinene;
β-myrcene;
β-pinene;
δ-limonene;
linalool;
β-caryophyllene;
α-humulene;
guaiol;
caryophyllene oxide; and/or
α-bisabolol.

In an embodiment, provided herein is a hemp extract comprising:
α-pinene;
β-myrcene;
β-pinene;
δ-limonene;
linalool;
β-caryophyllene;
α-humulene;
guaiol;
caryophyllene oxide; and
α-bisabolol.

In an embodiment, provided herein is a hemp extract comprising one or more of
β-myrcene;
δ-limonene;
cis-β-ocimene;
linalool;
β-caryophyllene;
α-humulene;
guaiol;
caryophyllene oxide; and/or
α-bisabolol.

In an embodiment, provided herein is a hemp extract comprising:
β-myrcene;
δ-limonene;
cis-β-ocimene;
linalool;
β-caryophyllene;
α-humulene;
guaiol;
caryophyllene oxide; and
α-bisabolol.

In an embodiment, the ratio of cannabidiol to cannabidiolic acid is about 0.1:1 to about 1:0.1. In another embodiment, the ratio of cannabidiol to cannabidiolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabidiol to cannabidiolic acid is about 0.6:1 to about 1:0.6. In still another embodiment, the ratio of cannabidiol to cannabidiolic acid is about 1:1.

In an embodiment, the concentration of Δ9-hydrocannabinol is insufficient to produce a psychotropic effect. In another embodiment, the ratio of Δ9-tetrahydrocannabinol to the other cannabinoids is from about 1:50 to about 1:15. In yet another embodiment, the ratio of Δ9-tetrahydrocannabinol to the other cannabinoids is about 1:50. In still another embodiment, the ratio of Δ9-tetrahydrocannabinol to the other cannabinoids is about 1:45. In an embodiment, the ratio of Δ9-tetrahydrocannabinol to the other cannabinoids is about 1:40. In another embodiment, the ratio of Δ9-tetrahydrocannabinol to the other cannabinoids is about 1:35. In yet another embodiment, the ratio of Δ9-tetrahydrocannabinol to the other cannabinoids is about 1:30. In still another embodiment, the ratio of Δ9-tetrahydrocannabinol to the other cannabinoids is about 1:25. In an embodiment, the ratio of Δ9-tetrahydrocannabinol to the other cannabinoids is about 1:20. In an embodiment, the ratio of Δ9-tetrahydrocannabinol to the other cannabinoids is about 1:15.

In an embodiment, the concentration of Δ9-tetrahydrocannabinol is less than about 7 mg/mL. In an embodiment, the concentration of Δ9-tetrahydrocannabinol is less than about 6 mg/mL. In an embodiment, the concentration of Δ9-tetrahydrocannabinol is less than about 5 mg/mL. In an embodiment, the concentration of Δ9-tetrahydrocannabinol is less than about 4 mg/mL. In an embodiment, the concentration of Δ9-tetrahydrocannabinol is less than about 3 mg/mL. In an embodiment, the concentration of Δ9-tetrahydrocannabinol is less than about 2 mg/mL. In another embodiment, the concentration of Δ9-tetrahydrocannabinol is less than about 1.5 mg/mL. In yet another embodiment, the concentration of Δ9-tetrahydrocannabinol is less than about 1 mg/mL. In still another embodiment, the concentration of Δ9-tetrahydrocannabinol is less than about 0.9 mg/mL. In yet another embodiment, the concentration of Δ9-tetrahydrocannabinol is less than about 0.8 mg/mL. In an embodiment, the concentration of Δ9-tetrahydrocannabinol is less than about 0.7 mg/mL. In another embodiment, the concentration of Δ9-tetrahydrocannabinol is less than about 0.6 mg/mL. In yet another embodiment, the concentration of Δ9-tetrahydrocannabinol is less than about 0.5 mg/mL. In still another embodiment, the concentration of Δ9-tetrahydrocannabinol is less than about 0.4 mg/mL. In an embodiment, the concentration of Δ9-tetrahydrocannabinol is less than about 0.3 mg/mL. In another embodiment, the concentration of Δ9-tetrahydrocannabinol is less than about 0.2 mg/mL. In yet another embodiment, the concentration of Δ9-tetrahydrocannabinol is less than about 0.1 mg/mL. In another embodiment, the concentration of Δ9-tetrahydrocannabinol is less than about 0.05 mg/mL. In another embodiment, the concentration of Δ9-tetrahydrocannabinol is about 0 mg/mL.

In an embodiment, the hemp extract does not comprise terpenes.

In an embodiment, the hemp extract comprises 1 or more of the following terpenes: α-pinene; β-myrcene; β-pinene; δ-limonene; linalool; β-caryophyllene; α-humulene; guaiol; caryophyllene oxide; α-bisabolol; and cis-β-ocimene.

In an embodiment, the hemp extract comprises 2 or more of the following: α-pinene; β-myrcene; β-pinene; δ-limonene; linalool; β-caryophyllene; α-humulene; guaiol; caryophyllene oxide; α-bisabolol; and cis-β-ocimene.

In an embodiment, the hemp extract comprises 3 or more of the following: α-pinene; β-myrcene; β-pinene; δ-limonene; linalool; β-caryophyllene; α-humulene; guaiol; caryophyllene oxide; α-bisabolol; and cis-β-ocimene.

In an embodiment, the hemp extract comprises 4 or more of the following: α-pinene; β-myrcene; β-pinene; δ-limonene; linalool; β-caryophyllene; α-humulene; guaiol; caryophyllene oxide; α-bisabolol; and cis-β-ocimene.

In an embodiment, the hemp extract comprises 5 or more of the following: α-pinene; β-myrcene; β-pinene; δ-limonene; linalool; β-caryophyllene; α-humulene; guaiol; caryophyllene oxide; α-bisabolol; and cis-β-ocimene.

In an embodiment, the hemp extract comprises 6 or more of the following: α-pinene; β-myrcene; β-pinene; δ-limonene; linalool; β-caryophyllene; α-humulene; guaiol; caryophyllene oxide; α-bisabolol; and cis-β-ocimene.

In an embodiment, the hemp extract comprises 7 or more of the following: α-pinene; β-myrcene; β-pinene; δ-limonene; linalool; β-caryophyllene; α-humulene; guaiol; caryophyllene oxide; α-bisabolol; and cis-β-ocimene.

In an embodiment, the hemp extract comprises 8 or more of the following: α-pinene; β-myrcene; β-pinene; δ-limonene; linalool; β-caryophyllene; α-humulene; guaiol; caryophyllene oxide; α-bisabolol; and cis-β-ocimene.

In an embodiment, the hemp extract comprises 9 or more of the following: α-pinene; β-myrcene; β-pinene; δ-limonene; linalool; β-caryophyllene; α-humulene; guaiol; caryophyllene oxide; α-bisabolol; and cis-β-ocimene.

In an embodiment, the hemp extract comprises 10 or more of the following: α-pinene; β-myrcene; β-pinene; δ-limonene; linalool; β-caryophyllene; α-humulene; guaiol; caryophyllene oxide; α-bisabolol; and cis-β-ocimene.

In an embodiment, the hemp extract comprises the following: α-pinene; β-myrcene; 3-pinene; δ-limonene; linalool; β-caryophyllene; α-humulene; guaiol; caryophyllene oxide; α-bisabolol; and cis-β-ocimene.

In an embodiment, the composition is formulated with a carrier. In another embodiment, the carrier is selected from the group consisting of hemp seed oil, linseed oil, olive oil, fish oil, salmon oil, coconut oil, catnip oil, sesame oil, MCT oil, lecithin, NF-971P, and grapeseed oil.

In an embodiment, the carrier comprises hemp seed oil.

In an embodiment, the carrier comprises sesame oil.

In an embodiment, the carrier comprises lecithin. In another embodiment, the lecithin is sunflower lecithin. In another embodiment, the sunflower lecithin is about 5%, about 10%, about 15%, about 20%, about 25%, about 30%, about 35%, about 40%, about 45%, or 50% (e.g., weight percent).

In an embodiment, the pharmaceutical composition comprises NF-971P polymer. In an embodiment, the NF-971P polymer is about 0.5%, about 1.0%, about 1.5%, about 2.0%, about 2.5%, or about 3.0% weight/volume ratio. In some embodiments, the NF-971P polymer comprises carbomer homopolymer.

In some embodiments, a hemp extract disclosed herein is incorporated into a pharmaceutical composition. In some embodiments, the pharmaceutical composition is formulated as a sublingual spray. In still another embodiment, the pharmaceutical composition is formulated as a water or alcohol soluble solution, a gel, or a cream for topical or transdermal application. In an embodiment, the pharmaceutical composition is applied to the back of the neck. In an embodiment, the pharmaceutical composition is applied via transdermal aural application. In an embodiment, the pharmaceutical composition is formulated as a gel for buccal or mucosal administration. In an embodiment, the pharmaceutical composition is formulated as a paste for buccal or mucosal administration. In another embodiment, the pharmaceutical composition is formulated as a solution for subcutaneous injection. In yet another embodiment, the pharmaceutical composition is formulated as a tablet. In still another embodiment, the pharmaceutical composition is formulated as a capsule. In an embodiment, the pharmaceutical composition is formulated as a hard chewable. In an embodiment, the pharmaceutical composition is formulated as a soft chewable.

In an embodiment, the composition is formulated as a chew for oral administration. In another embodiment, the chew is produced using cold extrusion. In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

The pharmaceutical compositions of the present disclosure may be manufactured by processes well known in the art, e.g., by means of conventional mixing, dissolving, granulating, grinding, pulverizing, dragee-making, levigating, emulsifying, encapsulating, entrapping or by lyophilizing processes.

The compositions for use in accordance with the present disclosure thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

### Dosage Forms

In an aspect, provided herein is a dosage form comprising:
a hemp extract disclosed herein and
and at least one of a pharmaceutically acceptable additive, a flavoring agent, a surfactant, and an adjuvant.

In an embodiment, the dosage form is formulated as a sublingual spray. In an embodiment, the dosage form is formulated as a water, alcohol, polyethylene glycol, or glycerol soluble solution or cream for topical or transdermal application. In an embodiment, the dosage form is formulated as a gel for buccal or mucosal administration. In an embodiment, the dosage form is formulated as a paste for buccal or mucosal administration. In an embodiment, the dosage form is formulated as a gel. In an embodiment, the dosage form is formulated as a tablet. In an embodiment, the dosage form is formulated as a capsule. In an embodiment, the dosage form is formulated as a hard chewable. In an embodiment, the dosage form is formulated as a soft chewable. In an embodiment, the dosage form is formulated for administration using a nebulizer. In an embodiment, the dosage form is formulated for inhalation. In an embodiment, the dosage form is formulated for administration using a pet collar. In an embodiment, the composition is formulated as an edible product for oral administration. In an embodiment, the dosage form is formulated as a chew for oral administration. In an embodiment, the chew is produced using cold extrusion.

In still another embodiment, the dosage form is formulated as a water or alcohol soluble solution, a gel, or a cream for topical or transdermal application. In an embodiment, the dosage form is applied to the back of the neck. In an embodiment, the dosage form is applied via transdermal aural application. In another embodiment, the dosage form is administered at a dose of 4 mg/kg. In another embodiment, the dosage form is administered twice daily for four weeks. In an embodiment, the dosage form is formulated as a gel for buccal or mucosal administration. In some embodiments, the dosage form is formulated as a paste for buccal or mucosal administration. In an embodiment, the dosage form is formulated as a powder. In another embodiment, the dosage form is formulated as a solution for subcutaneous injection. In yet another embodiment, the dosage form is formulated as a tablet. In still another embodiment, the dosage form is formulated as a capsule. In an embodiment, the dosage form is formulated as a soft chewable.

In some embodiments, the invention includes infusing edible products with hemp extract. In another embodiment, the edible product is an extruded food product, baked food product, nut butter, spread, pelleted feed, or processed food. In another embodiment, the edible product is a pet food. In another embodiment the edible product is in a dry, shelf-stable form such as dried fish, dried dairy products, fish meal, fish flour, cereals, flours, carbohydrates, dried fruits, etc. In another embodiment, the edible product is moist or semi-moist. In another embodiment, the edible product contains additives or supplements such as vitamins, minerals, medicinals, etc., for example chemicals, enzymes, etc., capable of removing plaque or tartar from the animal's teeth, etc. In an embodiment, the hemp extract is administered with catnip oil. In another embodiment, any of the dosage forms described can also include catnip.

In another embodiment, hemp extracts are administered using a nebulizer. In another embodiment, the nebulizer delivery device and system is capable of effectively and efficiently administering one or more nebulized drug to an animal. In another embodiment, the nebulizer system can easily be used on animals without removing them from their natural environment. In another embodiment, the nebulizer delivery device and system enables animals to be easily treated daily or multiple times a day without undue stress or the need for extensive resources. In another embodiment, the nebulizer delivery device and system can be used on animals having varying levels of training.

In one embodiment, hemp extract is administered using a diffuser. The diffuser can be any device which disperses hemp extract into the air. Hemp extract may be dispersed by any method, including by natural convection, by forced convection, by heating a wick or pad, for example, holding the hemp extract, by using pumps, or with fans.

In one embodiment, hemp extract is administered by a pet collar. The pet collar may comprise a belt with a buckle on one side, a free end on the other side and an attachment means, such as apertures disposed longitudinally within the central portion of the belt, or a quick release clasp mechanism, for securing the collar in a closed loop configuration. The pet collar may be made from a variety of materials including nylon, polyester leather or other suitable material. The belt material may be treated with a water-proofing compound. The nylon or polyester belt may be interwoven with reflective fibers to enhance the visibility of the pet collar during nighttime hours. In one embodiment, the collar is infused with hemp extract.

In an embodiment, the dosage form is formulated as a chew for oral administration. In another embodiment, the chew is produced using cold extrusion. In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

### Methods of Treatment

In an aspect, provided herein is a method for treating a disease, disorder, syndrome, and/or condition in a subject in need thereof, comprising administering to the subject a therapeutically effective amount of any of the hemp extracts or dosage forms described above. In an embodiment, the subject suffers from inflammation, anxiety, sleep disorders, pain (e.g., chronic pain, non-chronic pain, neuropathic pain, neurological dysfunction pain, nociceptive pain, post-operation pain), seizure, epilepsy, osteoarthritis, atopy, allergies, diarrhea (e.g., idiopathic diarrhea), skin wounds, gastrointestinal conditions, behavioral issues, obsessive behaviors, inflammatory bowel disease, dermatological conditions (e.g., pruritus, pyoderma), or anxiety.

In an embodiment, the dosage form or hemp extract is administered at a dosage of about 0.1-100.0 mg/kg. In an embodiment, the dosage form or hemp extract is administered at a dosage of about 1-100.0 mg/kg. In an embodiment, the dosage form or hemp extract is administered at a dosage of about 5-80.0 mg/kg. In an embodiment, the dosage form or hemp extract is administered at a dosage of about 5-80 mg/kg. In an embodiment, the dosage form or hemp extract is administered at a dosage of about 5-60.0 mg/kg. In an embodiment, the dosage form or hemp extract is administered at a dosage of about 7.5-60.0 mg/kg. In an embodiment, the dosage form or hemp extract is administered at a dosage of about 10-60.0 mg/kg. In an embodiment, the dosage form or hemp extract is administered at a dosage of about 5-50 mg/kg. In an embodiment, the dosage form or hemp extract is administered at a dosage of about 10-50.0 mg/kg. In an embodiment, the dosage form or hemp extract is administered at a dosage of about 15-60.0 mg/kg. In an embodiment, the dosage form or hemp extract is administered at a dosage of about 30-60.0 mg/kg. In an embodiment, the dosage form or hemp extract is administered at a dosage of about 15-50.0 mg/kg. In an embodiment, the dosage form or hemp extract is administered at a dosage of about 30-50.0 mg/kg. In an embodiment, the dosage form or hemp extract is administered at a dosage of about 20-35 mg/kg. In an embodiment, the dosage form or hemp extract is administered at a dosage of about 25-35 mg/kg. In an embodiment, the dosage form or hemp extract is administered at a dosage of about 35-50.0 mg/kg. In an embodiment, the dosage form or hemp extract is administered at a dosage of about 35-60.0 mg/kg. In an embodiment, the dosage form or hemp extract is administered at a dosage of about 0.1-15.0 mg/kg. In another embodiment, the dosage form or hemp extract is administered at a dosage of about 0.1-10.0 mg/kg. In another embodiment, the dosage form or hemp extract is administered at a dosage of about 1-10.0 mg/kg. In another embodiment, the dosage form or hemp extract is administered at a dosage of about 5-10.0 mg/kg. In another embodiment, the dosage form or hemp extract is administered at a dosage of about 7.5-10.0 mg/kg. In another embodiment, the dosage form or hemp extract is administered at a dosage of about 7.5-20.0 mg/kg. In another embodiment, the dosage form or hemp extract is administered at a dosage of about 10-30 mg/kg. In another embodiment, the dosage form or hemp extract is administered at a dosage of about 30-100 mg/kg. In an embodiment, the dosage is given orally. In an embodiment, the dosage is given topically.

In yet another embodiment, the dosage form or hemp extract is administered at a dosage of about 0.1 mg/kg. In still another embodiment, the dosage form or hemp extract is administered at a dosage of about 0.2 mg/kg. In yet another embodiment, the dosage form or hemp extract is administered at a dosage of about 0.3 mg/kg. In an embodiment, the dosage form or hemp extract is administered at a dosage of about 0.4 mg/kg. In another embodiment, the dosage form or hemp extract is administered at a dosage of about 0.5 mg/kg. In yet another embodiment, the dosage form or hemp extract is administered at a dosage of about 0.6 mg/kg. In still another embodiment, the dosage form or hemp extract is administered at a dosage of about 0.7 mg/kg. In yet another embodiment, the dosage form or hemp extract is administered at a dosage of about 0.8 mg/kg. In an embodiment, the dosage form or hemp extract is administered at a dosage of about 0.9 mg/kg. In another embodiment, the dosage form or hemp extract is administered at a dosage of about 1 mg/kg. In yet another embodiment, the dosage form or hemp extract is administered at a dosage of about 1.5 mg/kg. In still another embodiment, the dosage form or hemp extract is administered at a dosage of about 2 mg/kg. In an embodiment, the dosage form or hemp extract is administered at a dosage of about 3 mg/kg. In another embodiment, the dosage form or hemp extract is administered at a dosage of about 4 mg/kg. In yet another embodiment, the dosage form or hemp extract is administered at a dosage of about 5 mg/kg. In still another embodiment, the dosage form or hemp extract is administered at a dosage of about 6 mg/kg. In an embodiment, the dosage form or hemp extract is administered at a dosage of about 7 mg/kg. In an embodiment, the dosage form or hemp extract is administered at a dosage of about 7.5 mg/kg. In another embodiment, the dosage form or hemp extract is administered at a dosage of about 8 mg/kg. In yet another embodiment, the dosage form or hemp extract is administered at a dosage of about 9 mg/kg. In still another embodiment, the dosage form or hemp extract is administered at a dosage of about 10 mg/kg. In an embodiment, the dosage form or hemp extract is administered at a dosage of about 11 mg/kg. In another embodiment, the dosage form or hemp extract is administered at a dosage of about 12 mg/kg. In yet another embodiment, the dosage form or hemp extract is administered at a dosage of about 13 mg/kg. In still another embodiment, the dosage form or hemp extract is administered at a dosage of about 14 mg/kg. In an embodiment, the dosage form or hemp extract is administered at a dosage of about 15 mg/kg. In an embodiment, the dosage form or hemp extract is administered at a dosage of about 20 mg/kg. In an embodiment, the dosage form or hemp extract is administered at a dosage of about 25 mg/kg. In an embodiment, the dosage form or hemp extract is administered at a dosage of about 30 mg/kg. In an embodiment, the dosage form or hemp extract is administered at a dosage of about 35 mg/kg. In an embodiment, the dosage form or hemp extract is administered at a dosage of about 40 mg/kg. In an embodiment, the dosage form or hemp extract is administered at a dosage of about 45 mg/kg. In an embodiment, the dosage form or hemp extract is administered at a dosage of about 50 mg/kg. In an embodiment, the dosage form or hemp extract is administered at a dosage of about 60 mg/kg. In an embodiment, the dosage form or hemp extract is administered at a dosage of about 70 mg/kg. In an embodiment, the dosage form or hemp extract is administered at a dosage of about 80 mg/kg. In an embodiment, the dosage form or hemp extract is administered at a dosage of about 90 mg/kg. In an embodiment, the dosage form or hemp extract is administered at a dosage of about 100 mg/kg. In an embodiment, the dosage form or hemp extract is administered at a dosage of about 110 mg/kg. In an embodiment, the dosage is given orally. In an embodiment, the dosage is given topically.

In some embodiments, the hemp extract or dosage form is administered at a dosage selected from the group consisting of: 5 mg/kg, 7.5 mg/kg, 10 mg/kg, 20 mg/kg, 50 mg/kg, and 60 mg/kg.

In some embodiments, the hemp extract or dosage form is administered at a dosage of 5 mg/kg to 60 mg/kg.

In some embodiments, the hemp extract or dosage form is administered at a dosage of 10 mg/kg to 100 mg/kg.

In another embodiment, the dosage form or hemp extract is administered at twice the therapeutically effective dosage for one week, and then subsequently administered at a therapeutically effective dosage. In yet another embodiment, the therapeutically effective dosage is about 0.1-0.5 mg/kg. In still another embodiment, the therapeutically effective dosage is about 2 mg/kg. In an embodiment, the therapeutically effective dosage is about 8 mg/kg.

In an embodiment, the dosage form or hemp extract is administered at a dosage of about 1 mg/kg for one week, and then subsequently administered at a dosage of about 0.1-0.5 mg/kg. In another embodiment, the dosage form or hemp extract is administered at a dosage of about 4 mg/kg for one week, and then subsequently administered at a dosage of about 2 mg/kg.

In an embodiment, the dosage form or hemp extract is administered at a dosage of about 1.0 mg/kg once daily. In an embodiment, the dosage form or hemp extract is administered at a dosage of about 1.0 mg/kg twice daily. In an embodiment, the dosage form or hemp extract is administered at a dosage of about 1.0 mg/kg three times daily. In an embodiment, the dosage form or hemp extract is administered at a dosage of about 1.0 mg/kg four times daily.

In an embodiment, the dosage form or hemp extract is administered at a dosage of about 2.0 mg/kg once daily. In an embodiment, the dosage form or hemp extract is administered at a dosage of about 2.0 mg/kg twice daily. In an embodiment, the dosage form or hemp extract is administered at a dosage of about 2.0 mg/kg three times daily. In an embodiment, the dosage form or hemp extract is administered at a dosage of about 2.0 mg/kg four times daily.

In an embodiment, the dosage form or hemp extract is administered at a dosage of about 3.0 mg/kg once daily. In an embodiment, the dosage form or hemp extract is administered at a dosage of about 3.0 mg/kg twice daily. In an embodiment, the dosage form or hemp extract is administered at a dosage of about 3.0 mg/kg three times daily. In an embodiment, the dosage form or hemp extract is administered at a dosage of about 3.0 mg/kg four times daily.

In an embodiment, the dosage form or hemp extract is administered at a dosage of about 4.0 mg/kg once daily. In an embodiment, the dosage form or hemp extract is administered at a dosage of about 4.0 mg/kg twice daily. In an embodiment, the dosage form or hemp extract is administered at a dosage of about 4.0 mg/kg three times daily. In an embodiment, the dosage form or hemp extract is administered at a dosage of about 4.0 mg/kg four times daily. In an embodiment, the dosage form or hemp extract is administered at a dosage of about 5.0 mg/kg once daily. In an embodiment, the dosage form or hemp extract is administered at a dosage of about 5.0 mg/kg twice daily. In an embodiment, the dosage form or hemp extract is administered at a dosage of about 5.0 mg/kg three times daily. In an embodiment, the dosage form or hemp extract is administered at a dosage of about 5.0 mg/kg four times daily.

In an embodiment, the dosage form or hemp extract is administered at a dosage of about 6.0 mg/kg once daily. In an embodiment, the dosage form or hemp extract is administered at a dosage of about 6.0 mg/kg twice daily. In an embodiment, the dosage form or hemp extract is administered at a dosage of about 6.0 mg/kg three times daily. In an embodiment, the dosage form or hemp extract is administered at a dosage of about 6.0 mg/kg four times daily.

In an embodiment, the dosage form or hemp extract is administered at a dosage of about 7.0 mg/kg once daily. In an embodiment, the dosage form or hemp extract is administered at a dosage of about 7.0 mg/kg twice daily. In an embodiment, the dosage form or hemp extract is administered at a dosage of about 7.0 mg/kg three times daily. In an embodiment, the dosage form or hemp extract is administered at a dosage of about 7.0 mg/kg four times daily.

In an embodiment, the dosage form or hemp extract is administered at a dosage of about 8.0 mg/kg once daily. In an embodiment, the dosage form or hemp extract is administered at a dosage of about 8.0 mg/kg twice daily. In an embodiment, the dosage form or hemp extract is administered at a dosage of about 8.0 mg/kg three times daily. In an embodiment, the dosage form or hemp extract is administered at a dosage of about 8.0 mg/kg four times daily.

In an embodiment, the dosage form or hemp extract is administered at a dosage of about 9.0 mg/kg once daily. In an embodiment, the dosage form or hemp extract is administered at a dosage of about 9.0 mg/kg twice daily. In an embodiment, the dosage form or hemp extract is administered at a dosage of about 9.0 mg/kg three times daily. In an embodiment, the dosage form or hemp extract is administered at a dosage of about 9.0 mg/kg four times daily.

In an embodiment, the dosage form or hemp extract is administered at a dosage of about 10.0 mg/kg once daily. In an embodiment, the dosage form or hemp extract is administered at a dosage of about 10.0 mg/kg twice daily. In an embodiment, the dosage form or hemp extract is administered at a dosage of about 10.0 mg/kg three times daily. In an embodiment, the dosage form or hemp extract is administered at a dosage of about 10.0 mg/kg four times daily.

In an embodiment, the dosage form or hemp extract is administered at a dosage of about 2 mg/kg twice daily.

In an embodiment, a dropperful of the dosage form or hemp extract is administered to the subject. In another embodiment, 0.5 mL of the dosage form or hemp extract is administered to the subject. In another embodiment, 1 mL of the dosage form or hemp extract is administered to the subject. In another embodiment, 2 mL of the dosage form or hemp extract is administered to the subject. In another embodiment, 3 mL of the dosage form or hemp extract is administered to the subject. In another embodiment, 4 mL of the dosage form or hemp extract is administered to the subject. In another embodiment, 5 mL of the dosage form or hemp extract is administered to the subject. In another embodiment, 6 mL of the dosage form or hemp extract is administered to the subject. In another embodiment, 7 mL of the dosage form or hemp extract is administered to the subject. In another embodiment, 8 mL of the dosage form or hemp extract is administered to the subject. In another embodiment, 9 mL of the dosage form or hemp extract is administered to the subject. In another embodiment, 10 mL of the dosage form or hemp extract is administered to the subject.

In an embodiment, the method results in a therapeutically effective median maximal serum concentration of cannabidiol (CBD). In another embodiment, the median maximal serum concentration of CBD is about 30-90 ng/mL. In another embodiment, the median maximal serum concentration of CBD is about 30 ng/mL. In another embodiment, the median maximal serum concentration of CBD is about 50 ng/mL. In another embodiment, the median maximal serum concentration of CBD is about 70 ng/mL. In another embodiment, the median maximal serum concentration of CBD is about 90 ng/mL. In another embodiment, the median maximal serum concentration of CBD is about 90-310 ng/mL. In yet another embodiment, the median maximal serum concentration of CBD is about 90 ng/mL. In still another embodiment, the median maximal serum concentration of CBD is about 100 ng/mL. In still another embodiment, the median maximal serum concentration of CBD is about 102 ng/mL. In an embodiment, the median maximal serum concentration of CBD is about 200 ng/mL. In another embodiment, the median maximal serum concentration of CBD is about 300 ng/mL. In yet another embodiment, the median maximal serum concentration of CBD is about 400 ng/mL. In still another embodiment, the median maximal serum concentration of CBD is about 500 ng/mL. In an embodiment, the median maximal serum concentration of CBD is about 590 ng/mL. In another embodiment, the median maximal serum concentration of CBD is about 600 ng/mL.

In an embodiment, the method results in a therapeutically effective median maximal serum concentration of cannabidiolic acid (CBDA). In another embodiment, the median maximal serum concentration of CBDA is about 30-90 ng/mL. In another embodiment, the median maximal serum concentration of CBDA is about 30 ng/mL. In another embodiment, the median maximal serum concentration of CBDA is about 50 ng/mL. In another embodiment, the median maximal serum concentration of CBDA is about 70 ng/mL. In another embodiment, the median maximal serum concentration of CBDA is about 90 ng/mL. In another embodiment, the median maximal serum concentration of CBDA is about 90-310 ng/mL. In yet another embodiment, the median maximal serum concentration of CBDA is about 90 ng/mL. In still another embodiment, the median maximal serum concentration of CBDA is about 100 ng/mL. In still another embodiment, the median maximal serum concentration of CBDA is about 102 ng/mL. In an embodiment, the median maximal serum concentration of CBDA is about 200 ng/mL. In another embodiment, the median maximal serum concentration of CBDA is about 300 ng/mL. In yet another embodiment, the median maximal serum concentration of CBDA is about 400 ng/mL. In still another embodiment, the median maximal serum concentration of CBDA is about 500 ng/mL. In an embodiment, the median maximal serum concentration of CBDA is about 590 ng/mL. In another embodiment, the median maximal serum concentration of CBDA is about 600 ng/mL.

In an embodiment, the method results in a therapeutically effective median maximal serum concentration of CBD and CBDA. In another embodiment, the median maximal serum concentration of CBD and CBDA is about 30-90 ng/mL. In another embodiment, the median maximal serum concentration of CBD and CBDA is about 30 ng/mL. In another embodiment, the median maximal serum concentration of CBD and CBDA is about 50 ng/mL. In another embodiment, the median maximal serum concentration of CBD and CBDA is about 70 ng/mL. In another embodiment, the median maximal serum concentration of CBD and CBDA is about 90 ng/mL. In another embodiment, the median maximal serum concentration of CBD and CBDA is about 90-310 ng/mL. In yet another embodiment, the median maximal serum concentration of CBD and CBDA is about 90 ng/mL. In still another embodiment, the median maximal serum concentration of CBD and CBDA is about 100 ng/mL. In still another embodiment, the median maximal serum concentration of CBD and CBDA is about 102 ng/mL. In an embodiment, the median maximal serum concentration of CBD and CBDA is about 200 ng/mL. In another embodiment, the median maximal serum concentration of CBD and CBDA is about 300 ng/mL. In yet another embodiment, the median maximal serum concentration of CBD and CBDA is about 400 ng/mL. In still another embodiment, the median maximal serum concentration of CBD and CBDA is about 500 ng/mL. In an embodiment, the median maximal serum concentration of CBD and CBDA is about 590 ng/mL. In another embodiment, the median maximal serum concentration of CBD and CBDA is about 600 ng/mL.

In an embodiment, the method results in a therapeutically effective median maximal serum concentration of cannabigerol (CBG). In another embodiment, the median maximal serum concentration of CBG is about 30-90 ng/mL. In another embodiment, the median maximal serum concentration of CBG is about 30 ng/mL. In another embodiment, the median maximal serum concentration of CBG is about 50 ng/mL. In another embodiment, the median maximal serum concentration of CBG is about 70 ng/mL. In another embodiment, the median maximal serum concentration of CBG is about 90 ng/mL. In another embodiment, the median maximal serum concentration of CBG is about 90-310 ng/mL. In yet another embodiment, the median maximal serum concentration of CBG is about 90 ng/mL. In still another embodiment, the median maximal serum concentration of CBG is about 100 ng/mL. In still another embodiment, the median maximal serum concentration of CBG is about 102 ng/mL. In an embodiment, the median maximal serum concentration of CBG is about 200 ng/mL. In another embodiment, the median maximal serum concentration of CBG is about 300 ng/mL. In yet another embodiment, the median maximal serum concentration of CBG is about 400 ng/mL. In still another embodiment, the median maximal serum concentration of CBG is about 500 ng/mL. In an embodiment, the median maximal serum concentration of CBG is about 590 ng/mL. In another embodiment, the median maximal serum concentration of CBG is about 600 ng/mL.

In an embodiment, the method results in a therapeutically effective median maximal serum concentration of cannabigerolic acid (CBGA). In another embodiment, the median maximal serum concentration of CBGA is about 30-90 ng/mL. In another embodiment, the median maximal serum concentration of CBGA is about 30 ng/mL. In another embodiment, the median maximal serum concentration of CBGA is about 50 ng/mL. In another embodiment, the median maximal serum concentration of CBGA is about 70 ng/mL. In another embodiment, the median maximal serum concentration of CBGA is about 90 ng/mL. In another embodiment, the median maximal serum concentration of CBGA is about 90-310 ng/mL. In yet another embodiment, the median maximal serum concentration of CBGA is about 90 ng/mL. In still another embodiment, the median maximal serum concentration of CBGA is about 100 ng/mL. In still another embodiment, the median maximal serum concentration of CBGA is about 102 ng/mL. In an embodiment, the median maximal serum concentration of CBGA is about 200 ng/mL. In another embodiment, the median maximal serum concentration of CBGA is about 300 ng/mL. In yet another embodiment, the median maximal serum concentration of CBGA is about 400 ng/mL. In still another embodiment, the median maximal serum concentration of CBGA is about 500 ng/mL. In an embodiment, the median maximal serum concentration of CBGA is about 590 ng/mL. In another embodiment, the median maximal serum concentration of CBGA is about 600 ng/mL.

In an embodiment, the method results in a therapeutically effective median maximal serum concentration of CBG and CBGA. In another embodiment, the median maximal serum concentration of CBG and CBGA is about 30-90 ng/mL. In another embodiment, the median maximal serum concentration of CBG and CBGA is about 30 ng/mL. In another embodiment, the median maximal serum concentration of CBG and CBGA is about 50 ng/mL. In another embodiment, the median maximal serum concentration of CBG and CBGA is about 70 ng/mL. In another embodiment, the median maximal serum concentration of CBG and CBGA is about 90 ng/mL. In another embodiment, the median maximal serum concentration of CBG and CBGA is about 90-310 ng/mL. In yet another embodiment, the median maximal serum concentration of CBG and CBGA is about 90 ng/mL. In still another embodiment, the median maximal serum concentration of CBG and CBGA is about 100 ng/mL. In still another embodiment, the median maximal serum concentration of CBG and CBGA is about 102 ng/mL. In an embodiment, the median maximal serum concentration of CBG and CBGA is about 200 ng/mL. In another embodiment, the median maximal serum concentration of CBG and CBGA is about 300 ng/mL. In yet another embodiment, the median maximal serum concentration of CBG and CBGA is about 400 ng/mL. In still another embodiment, the median maximal serum concentration of CBG and CBGA is about 500 ng/mL. In an embodiment, the median maximal serum concentration of CBG and CBGA is about 590 ng/mL. In another embodiment, the median maximal serum concentration of CBG and CBGA is about 600 ng/mL.

In an embodiment, the method results in a therapeutically effective median maximal serum concentration of cannabidivarin (CBDV). In another embodiment, the median maximal serum concentration of CBDV is about 30-90 ng/mL. In another embodiment, the median maximal serum concentration of CBDV is about 30 ng/mL. In another embodiment, the median maximal serum concentration of CBDV is about 50 ng/mL. In another embodiment, the median maximal serum concentration of CBDV is about 70 ng/mL. In another embodiment, the median maximal serum concentration of CBDV is about 90 ng/mL. In another embodiment, the median maximal serum concentration of CBDV is about 90-310 ng/mL. In yet another embodiment, the median maximal serum concentration of CBDV is about 90 ng/mL. In still another embodiment, the median maximal serum concentration of CBDV is about 100 ng/mL. In still another embodiment, the median maximal serum concentration of CBDV is about 102 ng/mL. In an embodiment, the median maximal serum concentration of CBDV is about 200 ng/mL. In another embodiment, the median maximal serum concentration of CBDV is about 300 ng/mL. In yet another embodiment, the median maximal serum concentration of CBDV is about 400 ng/mL. In still another embodiment, the median maximal serum concentration of CBDV is about 500 ng/mL. In an embodiment, the median maximal serum concentration of CBDV is about 590 ng/mL. In another embodiment, the median maximal serum concentration of CBDV is about 600 ng/mL.

In an embodiment, the method results in a therapeutically effective median maximal serum concentration of cannabidivarinic acid (CBDVA). In another embodiment, the median maximal serum concentration of CBDVA is about 30-90 ng/mL. In another embodiment, the median maximal serum concentration of CBDVA is about 30 ng/mL. In another embodiment, the median maximal serum concentration of CBDVA is about 50 ng/mL. In another embodiment, the median maximal serum concentration of CBDVA is about 70 ng/mL. In another embodiment, the median maximal serum concentration of CBDVA is about 90 ng/mL. In another embodiment, the median maximal serum concentration of CBDVA is about 90-310 ng/mL. In yet another embodiment, the median maximal serum concentration of CBDVA is about 90 ng/mL. In still another embodiment, the median maximal serum concentration of CBDVA is about 100 ng/mL. In still another embodiment, the median maximal serum concentration of CBDVA is about 102 ng/mL. In an embodiment, the median maximal serum concentration of CBDVA is about 200 ng/mL. In another embodiment, the median maximal serum concentration of CBDVA is about 300 ng/mL. In yet another embodiment, the median maximal serum concentration of CBDVA is about 400 ng/mL. In still another embodiment, the median maximal serum concentration of CBDVA is about 500 ng/mL. In an embodiment, the median maximal serum concentration of CBDVA is about 590 ng/mL. In another embodiment, the median maximal serum concentration of CBDVA is about 600 ng/mL.

In an embodiment, the method results in a therapeutically effective median maximal serum concentration of CBDV and CBDVA. In another embodiment, the median maximal serum concentration of CBDV and CBDVA is about 30-90 ng/mL. In another embodiment, the median maximal serum concentration of CBDV and CBDVA is about 30 ng/mL. In another embodiment, the median maximal serum concentration of CBDV and CBDVA is about 50 ng/mL. In another embodiment, the median maximal serum concentration of CBDV and CBDVA is about 70 ng/mL. In another embodiment, the median maximal serum concentration of CBDV and CBDVA is about 90 ng/mL. In another embodiment, the median maximal serum concentration of CBDV and CBDVA is about 90-310 ng/mL. In yet another embodiment, the median maximal serum concentration of CBDV and CBDVA is about 90 ng/mL. In still another embodiment, the median maximal serum concentration of CBDV and CBDVA is about 100 ng/mL. In still another embodiment, the median maximal serum concentration of CBDV and CBDVA is about 102 ng/mL. In an embodiment, the median maximal serum concentration of CBDV and CBDVA is about 200 ng/mL. In another embodiment, the median maximal serum concentration of CBDV and CBDVA is about 300 ng/mL. In yet another embodiment, the median maximal serum concentration of CBDV and CBDVA is about 400 ng/mL. In still another embodiment, the median maximal serum concentration of CBDV and CBDVA is about 500 ng/mL. In an embodiment, the median maximal serum concentration of CBDV and CBDVA is about 590 ng/mL. In another embodiment, the median maximal serum concentration of CBDV and CBDVA is about 600 ng/mL.

In an embodiment, the method results in a therapeutically effective median maximal serum concentration of cannabinol (CBN). In another embodiment, the median maximal serum concentration of CBN is about 30-90 ng/mL. In another embodiment, the median maximal serum concentration of CBN is about 30 ng/mL. In another embodiment, the median maximal serum concentration of CBN is about 50 ng/mL. In another embodiment, the median maximal serum concentration of CBN is about 70 ng/mL. In another embodiment, the median maximal serum concentration of CBN is about 90 ng/mL. In another embodiment, the median maximal serum concentration of CBN is about 90-310 ng/mL. In yet another embodiment, the median maximal serum concentration of CBN is about 90 ng/mL. In still another embodiment, the median maximal serum concentration of CBN is about 100 ng/mL. In still another embodiment, the median maximal serum concentration of CBN is about 102 ng/mL. In an embodiment, the median maximal serum concentration of CBN is about 200 ng/mL. In another embodiment, the median maximal serum concentration of CBN is about 300 ng/mL. In yet another embodiment, the median maximal serum concentration of CBN is about 400 ng/mL. In still another embodiment, the median maximal serum concentration of CBN is about 500 ng/mL. In an embodiment, the median maximal serum concentration of CBN is about 590 ng/mL. In another embodiment, the median maximal serum concentration of CBN is about 600 ng/mL.

In an embodiment, the subject is a veterinary subject. In an embodiment, the veterinary subject is canine, feline, bovine, porcine, or equine. In another embodiment, the veterinary subject is canine. In yet another embodiment, the veterinary subject is feline. In yet another embodiment, the veterinary subject is equine.

In another embodiment, the subject is human.

In an embodiment, the hemp extract is administered at a dosage of about 0.1-15.0 mg/kg. In another embodiment, the hemp extract is administered at a dosage of about 0.1-10.0 mg/kg. In yet another embodiment, the hemp extract is administered at a dosage of about 0.1 mg/kg. In still another embodiment, the hemp extract is administered at a dosage of about 0.2 mg/kg. In yet another embodiment, the hemp extract is administered at a dosage of about 0.3 mg/kg. In an embodiment, the hemp extract is administered at a dosage of about 0.4 mg/kg. In another embodiment, the hemp extract is administered at a dosage of about 0.5 mg/kg. In yet another embodiment, the hemp extract is administered at a dosage of about 0.6 mg/kg. In still another embodiment, the hemp extract is administered at a dosage of about 0.7 mg/kg. In yet another embodiment, the hemp extract is administered at a dosage of about 0.8 mg/kg. In an embodiment, the hemp extract is administered at a dosage of about 0.9 mg/kg. In another embodiment, the hemp extract is administered at a dosage of about 1 mg/kg. In yet another embodiment, the hemp extract is administered at a dosage of about 1.5 mg/kg. In still another embodiment, the hemp extract is administered at a dosage of about 2 mg/kg. In an embodiment, the hemp extract is administered at a dosage of about 3 mg/kg. In another embodiment, the hemp extract is administered at a dosage of about 4 mg/kg. In yet another embodiment, the hemp extract is administered at a dosage of about 5 mg/kg. In still another embodiment, the hemp extract is administered at a dosage of about 6 mg/kg. In an embodiment, the hemp extract is administered at a dosage of about 7 mg/kg. In another embodiment, the hemp extract is administered at a dosage of about 8 mg/kg. In yet another embodiment, the hemp extract is administered at a dosage of about 9 mg/kg. In still another embodiment, the hemp extract is administered at a dosage of about 10 mg/kg. In an embodiment, the hemp extract is administered at a dosage of about 11 mg/kg. In another embodiment, the hemp extract is administered at a dosage of about 12 mg/kg. In yet another embodiment, the hemp extract is administered at a dosage of about 13 mg/kg. In still another embodiment, the hemp extract is administered at a dosage of about 14 mg/kg. In an embodiment, the hemp extract is administered at a dosage of about 15 mg/kg.

In another embodiment, the hemp extract is administered at twice the therapeutically effective dosage for one week, and then subsequently administered at a therapeutically effective dosage. In yet another embodiment, the therapeutically effective dosage is about 0.1-0.5 mg/kg. In still another embodiment, the therapeutically effective dosage is about 2 mg/kg. In an embodiment, the therapeutically effective dosage is about 8 mg/kg.

In an embodiment, the hemp extract is administered at a dosage of about 1 mg/kg for one week, and then subsequently administered at a dosage of about 0.1-0.5 mg/kg. In another embodiment, the hemp extract is administered at a dosage of about 4 mg/kg for one week, and then subsequently administered at a dosage of about 2 mg/kg.

In an embodiment, the method results in a therapeutically effective median maximal serum concentration of cannabidiol. In another embodiment, the median maximal serum concentration of cannabidiol is about 90-310 ng/mL. In yet another embodiment, the median maximal serum concentration of cannabidiol is about 90 ng/mL. In still another embodiment, the median maximal serum concentration of cannabidiol is about 100 ng/mL. In still another embodiment, the median maximal serum concentration of cannabidiol is about 102 ng/mL. In an embodiment, the median maximal serum concentration of cannabidiol is about 200 ng/mL. In another embodiment, the median maximal serum concentration of cannabidiol is about 300 ng/mL. In yet another embodiment, the median maximal serum concentration of cannabidiol is about 400 ng/mL. In still another embodiment, the median maximal serum concentration of cannabidiol is about 500 ng/mL. In an embodiment, the median maximal serum concentration of cannabidiol is about 590 ng/mL. In another embodiment, the median maximal serum concentration of cannabidiol is about 600 ng/mL.

The pharmaceutical compositions and dosage forms of the present disclosure may be administered by any convenient route, for example, by infusion or bolus injection, by absorption through epithelial or mucocutaneous linings (e.g., oral mucosa, rectal and intestinal mucosa, etc.) and may be administered together with any other therapeutic agent. Administration can be systemic or local. In an embodiment, administration is topical. In another embodiment, topical administration is used to treat local pain. In another embodiment, the local pain is joint pain. In an embodiment, the veterinary subject is an animal>100 kg (e.g., a horse, cow, or pig).

The therapeutic compositions of the invention (e.g., hemp extracts) will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The composition and dose may vary depending upon the age, weight, and gender of a subject to be administered, target disease, disorder, syndrome, condition, route of administration, and the like. Various delivery systems are known and can be used to administer the pharmaceutical composition of the invention, e.g., encapsulation in liposomes, microparticles, microcapsules, receptor mediated endocytosis (see, e.g., Wu et al. (1987) J. Biol. Chem. 262:4429-4432). Methods of introduction include, but are not limited to, intradermal, intramuscular, intraperitoneal, intravenous, topical, transdermal, buccal, sublingual, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum, methyl cellulose, hydroxypropylmethyl-cellulose, sodium carbomethylcellulose, and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures.

The injectable preparations may include dosage forms for intravenous, subcutaneous, intracutaneous and intramuscular injections, local injection, drip infusions, etc. These injectable preparations may be prepared by methods publicly known. For example, the injectable preparations may be prepared, e.g., by dissolving, suspending or emulsifying the dosage form or hemp extract in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active components may be dissolved or suspended in suitable liquids, such as fatty oils, liquid paraffin, or liquid polyethylene glycols.

Alternatively, the composition may be in a powder form for constitution before use with a suitable vehicle, e.g., sterile, pyrogen-free water. The exact formulation, route of administration and dosage may be chosen by the physician familiar with the patient's condition. (See for example Fingl, et al., 1975, in “The Pharmacological Basis of Therapeutics”, Chapter I, p. 1). Depending on the severity and responsiveness of the condition treated, dosing can also be a single administration of a slow release composition, with course of treatment lasting from several days to several weeks or until cure is effected or diminution of the disease state is achieved.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, chews, etc. In certain embodiments, for the dosages provided above, they are administered in one serving of an edible product, e.g. 1 mg/kg of hemp extract provided in an individual product.

Hemp extract, dosage forms, and pharmaceutical compositions described herein can be packaged to provide one or more doses of hemp extract per package. Any suitable type of packaging can be used, including wrappers, pouches, boxes, tubs, cans, blister packs, and bags. Such packaging is convenient and accessible to consumers, enhances the consumer's ease of use, reduces the presence of pathogens, increases shelf life, and reduces spoilage. In an embodiment, the hemp extract, dosage form, or pharmaceutical composition is packaged to provide one or more doses of hemp extract per package. In an embodiment, the package is resealable. In some embodiments, the dosage form is edible. In some embodiments, the edible dosage form is formed into a flat shape that can be more easily divided. In some embodiments, this flat shape is a disk or cookie shape. In some embodiments, the edible dosage form includes indentations to show where the edible dosage form should be divided to provide specific dosages. In some embodiments, the edible dosage form comes in multiple pieces. In some embodiments, each of the multiple pieces provides a certain dosage. In some embodiments, a package contains 1, 2, 3, 4, 5, 6, 7, 8, 9, 10, 11, 12, 13, 14, 15, 16, 17, 18, 19, 20, 25, 30, 35, 40, 45, 50, or more pieces. In some embodiments, the package is resealable. In an embodiment, one dose of hemp extract is a therapeutically effective amount. In accordance with the methods disclosed herein, pharmaceutical formulations can be administered to the patient or subject using any acceptable device or mechanism. For example, the administration can be accomplished using a syringe and needle or with a reusable pen and/or autoinjector delivery device. The methods of the present invention include the use of numerous reusable pen and/or autoinjector delivery devices to administer a pharmaceutical formulation.

In an embodiment for non-human animal administration, the term “pharmaceutical” as used herein may be replaced by “veterinary.”

In some embodiments, the patient is subject before, during, and/or after treatment to CHOP chemotherapy. CHOP chemotherapy comprises for example, without limitation, a cyclophosphamide, doxorubicin, vincristine, and steroid (e.g., prednisolone).

Various exemplary hemp extracts are provided below in Tables 1 and 2, as well as several concentrated hemp extracts. Concentrated hemp extracts comprise the concentrations of cannabinoids shown.

Table 1 shows concentrations for compositions A-E. The numeral after A, B, C, D, or E indicates one of three different values for each cannabinoid. Each of the two values (e.g., A1 and A2) were obtained via separate chemical processes or calculations and all fall within the scope of the corresponding compositions. For example, each of compositions A1 and A2 fall within the scope of “composition A.”

Table 2 shows concentrations for compositions F-J. The numeral after F, G, H, I, or J indicates one of three different values for each cannabinoid. Each of the two values (e.g., F1, F2, and F3) were obtained via separate chemical processes or calculations and all fall within the scope of the corresponding compositions. For example, each of compositions F1, F2, and F3 fall within the scope of “composition F.”

TABLE 1
Exemplary Hemp Extracts

| Composition | CBDV | CBDVA | THCV | CBD | CBG | CBDA | CBGA | CBN | THC | Δ8-THC | CBC | THC-A | CBCA |
|---|---|---|---|---|---|---|---|---|---|---|---|---|---|
| A1 | 0.08 | 0.10 | 0.30 | 31.10 | 1.01 | 32.54 | 0.73 | ND | 1.69 | 0.04 | 1.53 | 1.75 | 1.71 |
| A2 | 0.14 | ND | ND | 30.80 | 0.78 | 31.70 | 1.30 | ND | 1.37 | ND | 1.40 | 1.35 | ND |
| B1 | 0.07 | 0.08 | 0.28 | 26.79 | 0.15 | 28.08 | 0.61 | 10.39 | 1.42 | 0.04 | 1.30 | 1.50 | 1.45 |
| B2 | ND | ND | ND | 27.00 | 0.66 | 27.50 | 1.08 | 10.10 | 1.14 | ND | 1.19 | 1.14 | ND |
| C1 | 15.12 | 11.18 | 0.96 | 20.02 | 3.48 | 15.38 | 7.93 | 0.09 | 1.15 | 0.10 | 1.43 | 1.68 | 1.31 |
| C2 | 15.36 | ND | 1.08 | 19.90 | 3.50 | 15.20 | 7.81 | ND | 0.88 | ND | 1.16 | 0.62 | ND |
| D1 | ND | 0.22 | 0.34 | 1.15 | 3.88 | 33.76 | 31.12 | ND | 0.53 | 0.04 | 0.07 | 1.82 | 2.42 |
| D2 | ND | ND | ND | 1.24 | 3.93 | 32.60 | 32.70 | ND | 0.26 | ND | 0.37 | 1.50 | ND |
| E1 | 1.16 | 0.90 | 0.26 | 1.98 | 30.88 | 1.00 | 33.59 | 0.03 | 0.82 | ND | 1.32 | 0.59 | 1.28 |
| E2 | 1.17 | ND | ND | 2.06 | 30.90 | 1.03 | 34.70 | ND | 0.47 | ND | 1.16 | 0.39 | ND |

Values are concentrations in mg/mL.
ND—A concentration is either not determined, or is below reliable levels for reporting.

TABLE 2
Exemplary Hemp Extracts

| Composition | CBDV | CBDVA | THCV | CBD | CBG | CBDA | CBGA | CBN | THC | Δ8-THC | CBC | THC-A | CBCA |
|---|---|---|---|---|---|---|---|---|---|---|---|---|---|
| F1 | 0.11 | 0.14 | 0.43 | 43.55 | 1.42 | 45.55 | 1.02 | 0.00 | 2.37 | 0.06 | 2.14 | 2.45 | 2.39 |
| F2 | 0.11 | 0.14 | 0.42 | 43.95 | 1.28 | 45.65 | 1.04 | 0.05 | 2.38 | ND | 2.12 | 2.30 | 2.22 |
| F3 | 0.20 | ND | ND | 47.10 | 1.04 | 47.40 | 1.66 | ND | 1.92 | ND | 1.96 | 1.88 | ND |
| G1 | 0.09 | 0.12 | 0.39 | 37.51 | 0.21 | 39.31 | 0.85 | 14.54 | 1.99 | 0.06 | 1.83 | 2.09 | 2.03 |
| G2 | 0.10 | 0.12 | 0.34 | 37.54 | 1.05 | 39.31 | 0.85 | 14.52 | 2.02 | ND | 1.81 | 1.97 | 1.84 |
| G3 | 0.12 | NT | ND | 38.90 | 0.90 | 40.60 | 1.40 | 14.70 | 1.63 | ND | 1.67 | 1.61 | ND |
| H1 | 21.17 | 15.65 | 1.34 | 28.03 | 4.87 | 21.53 | 11.10 | 0.12 | 1.61 | 0.14 | 2.00 | 2.35 | 1.83 |
| H2 | 21.82 | 16.24 | 1.33 | 28.39 | 4.79 | 22.12 | 11.53 | 0.08 | 1.36 | 0.13 | 1.92 | 2.12 | 1.71 |
| H3 | 22.60 | ND | 1.54 | 28.40 | 4.94 | 22.40 | 12.20 | ND | 1.26 | ND | 1.66 | 0.88 | ND |
| I1 | 0.00 | 0.30 | 0.48 | 1.61 | 5.44 | 47.27 | 43.57 | 0.00 | 0.74 | 0.06 | 0.09 | 2.55 | 3.38 |
| I2 | 0.05 | 0.28 | 0.39 | 1.60 | 4.48 | 47.17 | 44.55 | ND | 0.71 | 0.04 | 0.52 | 2.44 | 3.44 |
| I3 | ND | ND | ND | 1.73 | 4.79 | 48.50 | 49.00 | ND | 0.35 | ND | 0.50 | 2.15 | ND |
| J1 | 1.63 | 1.26 | 0.36 | 2.77 | 43.23 | 1.39 | 47.02 | 0.04 | 1.14 | 0.00 | 1.84 | 0.83 | 1.79 |
| J2 | 1.65 | 0.94 | 0.33 | 2.77 | 45.07 | 1.02 | 48.92 | 0.08 | 1.15 | 0.05 | 1.84 | 0.89 | 1.83 |
| J3 | 1.57 | ND | ND | 2.86 | 44.50 | 1.01 | 51.10 | ND | 0.76 | ND | 1.67 | 0.41 | ND |

Values are concentrations in mg/mL.
ND—A concentration is either not determined, or is below reliable levels for reporting.

### Concentrated CBD-CBDA Extract—A

CBD: 26-36 mg/mL
CBDA: 27-37 mg/mL
CBC, CBGA, CBDV, CBG, THCA: 1-10 mg/mL
Total Cannabinoids: about 68 mg/mL
THC<0.3%

### Concentrated CBD-CBDA Extract—B

CBD: about 31 mg/mL
CBDA: about 32 mg/mL
CBC, CBGA, CBDV, CBG, THCA: about 5 mg/mL
Total Cannabinoids: about 68 mg/mL
THC<0.3%
CBD:THC ratio of about 23:1
Comprising the following terpenes: alpha-pinene, beta-myrcene, and beta-caryophyllene

### Concentrated CBD-CBDA-CBN Extract—A

CBD: 22-32 mg/mL
CBDA: 22-32 mg/mL
CBN: 5-15 mg/mL
CBG, CBC, THCA, CBGA: 1-8 mg/mL
Total Cannabinoids: about 68 mg/mL
THC<0.3%

### Concentrated CBD-CBDA-CBN Extract—B

CBD: about 27 mg/mL
CBDA: about 27 mg/mL
CBN: about 10 mg/mL
CBG, CBC, THCA, CBGA: about 4 mg/mL
Total Cannabinoids: about 68 mg/mL
THC<0.3%
CBD:THC ratio of about 24:1
Comprising the following terpenes: alpha-pinene, beta-myrcene, and beta-caryophyllene

### Concentrated CBDV-CBDVA-CBD-CBDA Extract—A

CBD: 15-25 mg/mL
CBDA: 10-20 mg/mL
CBDV: 10-20 mg/mL
CBGA, CBG, THCV, CBG, CBC, THCA: 9-19 mg/mL
Total Cannabinoids: about 64 mg/mL
THC<0.3%

### Concentrated CBDV-CBDVA-CBD-CBDA Extract—B

CBD: about 20 mg/mL
CBDA: about 15 mg/mL
CBDV: about 15 mg/mL
CBGA, CBG, THCV, CBG, CBC, THCA: about 14 mg/mL
Total Cannabinoids: about 64 mg/mL
THC<0.2%
CBD:THC ratio of about 19:1
Comprising the following terpenes: beta-caryophyllene and alpha-humulene

### Concentrated CBGA-CBG Extract—A

CBG: 26-36 mg/mL
CBGA: 30-40 mg/mL
CBD, CBDV, CBC, THCA, CBDA: 3-9 mg/mL
Total Cannabinoids: about 72 mg/mL
THC<0.3%

### Concentrated CBGA-CBG Extract—B

CBG: about 31 mg/mL
CBGA: about 35 mg/mL
CBD, CBDV, CBC, THCA, CBDA: about 6 mg/mL
Total Cannabinoids: about 72 mg/mL
THC<0.1%
Comprising the following terpenes: beta-caryophyllene and alpha-humulene

### Concentrated CBGA-CBDA Extract—A

CBGA: 28-38 mg/mL
CBDA: 28-38 mg/mL
CBD, CBG, CBC, THCA: 3-11 mg/mL
Total Cannabinoids: about 73 mg/mL
THC<0.3%

### Concentrated CBGA-CBDA Extract—B

CBGA: about 33 mg/mL
CBDA: about 33 mg/mL
CBD, CBG, CBC, THCA: about 7 mg/mL
Total Cannabinoids: about 73 mg/mL
THC<0.2%
Comprising the following terpenes: alpha-pinene, beta-myrcene, and beta-caryophyllene

### EXAMPLES

While several experimental Examples are disclosed herein, these Examples are non-limiting.

### Example 1. Study Using an Inflammatory Bowel Disease Model in Rats

A study will be conducted to evaluate hemp extracts for potential anti-inflammatory and/or analgesic activity using an inflammatory bowel disease (IBD) model in the mouse: the Dextran Sulfate Sodium (DSS)-induced colitis model.

For the induction of colitis, normal drinking water will be replaced with a 4.5% solution of Dextran Sodium Sulfate (DSS) from Day 0 (8:00 AM) to Day 7 (5:00 PM). One group will receive normal tap water. Mice will be evaluated after 7 days of DSS treatment. DSS bottles will be changed on Day 3 and Day 5. The control group will receive tap water.

Each test hemp extract to be tested will be evaluated at 3 doses, administered p.o. b.i.d. from day 0 to day 6 and 60 minutes before the test on Day 7, and compared to a vehicle (e.g., carrier alone) control group.

Cimetidine (30 mg/kg) administered p.o. 60 minutes before each testing phase test will be used as analgesic comparison substance (except paw use). To respect the number of administration per animal, distilled water will be used for the chronic dosing.

The experiment will therefore include 15 groups. The mice will be evaluated at different time points:
Day 4: to observe effect of DSS
Day 7: to observe effects of test substance on DSS treatment
Day 10: to observe effect in absence of DSS
Day 14: to observe potential recovery

### Evaluation of Visceral Pain

### Tactile Allodynia Evaluation: Electronic Von Frey Test

The animals will be acclimated to the boxes at least 1 h before initiating behavioral testing. The animal is placed under an inverted acrylic plastic box on a grid floor. The tip of an electronic von Frey probe is the applied with increasing force upon abdomen. The force required to induce a response is automatically recorded. This procedure is carried out 3 times and the mean force is calculated.

### Changes in Behavior: Abnormal Postures and Eye Closure

Eye closure: for the opening and closing of the eyes, 5 scale grades will be set and scored: zero for complete opening (normal eyes, no pain) and 10 for completely shut (maximal pain), 5 for half-closed eyes, and 2 and 7 for the 2 intermediate positions between open and half-closed and between half-closed and closed, respectively.

Abnormal postures: an additional score will evaluate normal posture/appearance and abnormal posture/appearance indicative of pain: the presence or absence of the following signs will be reported: licking, writhing, sedation and rounded back (observation of each mouse for few seconds).

Bodyweight of the mice will be periodically evaluated.

### Assessment of the Severity of Colitis (Day 14)

Bodyweight and length of the colon will be measured. The number of surviving animals will be counted daily from Day 0 to Day 7 and the body weights, the presence of occult or gross blood in the feces and stool consistency will be recorded for each mouse. These indexes will be assigned a score of severity and will be used to calculate the average daily disease activity index (DAI) for each animal.

The DAI scores: the disease activity index is the combined scores of body weight loss, stool consistency and rectal bleeding divided by 3.

TABLE 3
DAI Scores

| | Score Weight loss (%) | | |
|---|---|---|---|
| | as compared | Stool | Occult/gross |
| scores | with day 1 | consistency | rectal bleeding |
| 0 | None | Normal | Normal |
| 1 | 1-5 | | |
| 2 | 5-10 | Loose stools | Hemoccult |
| 3 | 10-20 | | |
| 4 | >20 | Diarrhea | Gross bleeding |

### Terminal Blood and Tissue Collections

### Plasma Sampling

At the completion of testing, all animals will be placed under isoflurane anesthesia and approximately 500 μl of blood will be collected by cardiac puncture using a sterile disposable syringe. The blood samples will be immediately transferred into pre-labeled tube containing K2-EDTA. After sealing each tube, the blood samples will be manually agitated and stored on ice until centrifugation (within 30 minutes of sampling). The samples will be centrifuged at +4° C., at 3000 g, for 10 minutes. The entire resultant plasma obtained will be immediately transferred to 2 suitably labeled polypropylene tubes (2 aliquots of approximately 100 μl) and flash frozen in liquid nitrogen or dry ice. The tubes will be stored upright at approximately −80° C.

### GI Track and Liver Collection

In all mice, immediately after decapitation and exsanguination, GI track and liver will be weighted and rinsed in ice-cold physiological saline, and stored in 1000 neutral buffered formalin in separate adequate pre-labeled polypropylene vials (Safe capsule', 100 buffered Formol=formaldehyde at 40%) at room temperature and protected from light.

The samples will be subjected to histopathological analysis.

Experimental treatment groups are described in Table 4. Compositions (“Comp.”) referred to in the treatment column correspond to those described in Table 1. The test or vehicle formulations will be administered as a repeated oral dose by gavage (b.i.d.). The vehicle will be sesame oil.

TABLE 4
Experimental treatment groups

| | | | | Dose- | | Administration |
|---|---|---|---|---|---|---|
| | Number of | DSS | | level | Concentration | Volume |
| Group | animals | 5% | Treatment | (mg/kg) | (mg/mL) | (mL/kg) |
| 1 | 10 males | No | Vehicle | — | — | 10 |
| 2 | 10 males | Yes | Vehicle | — | — | 10 |
| 3 | 10 males | Yes | Comp. A | 7.5 | 0.75 | 10 |
| 4 | 10 males | Yes | Comp. A | 20 | 2 | 10 |
| 5 | 10 males | Yes | Comp. A | 50 | 6 | 10 |
| 6 | 10 males | Yes | Comp. C | 7.5 | 0.75 | 10 |
| 7 | 10 males | Yes | Comp. C | 20 | 2 | 10 |
| 8 | 10 males | Yes | Comp. C | 50 | 6 | 10 |
| 9 | 10 males | Yes | Comp. D | 7.5 | 0.75 | 10 |
| 10 | 10 males | Yes | Comp. D | 20 | 2 | 10 |
| 11 | 10 males | Yes | Comp. D | 50 | 6 | 10 |
| 12 | 10 males | Yes | Comp. E | 7.5 | 0.75 | 10 |
| 13 | 10 males | Yes | Comp. E | 20 | 2 | 10 |
| 14 | 10 males | Yes | Comp. E | 50 | 6 | 10 |
| 15 | 10 males | Yes | Cimetidine | 30 | 3 | 10 |

### Example 2. Study Using an Osteoarthritis Model in Rats

A study will be conducted to evaluate selected hemp extracts for potential analgesic activity using the mono iodoacetate (MIA)-induced chronic inflammatory pain model (osteoarthritis model) in rats.

The method to be used in the study detects analgesic activity in rats suffering from chronic osteoarthritis.

Osteoarthritis induction on day 0 is accomplished via injection of monosodium iodoacetate (MIA) through the infra-patellar ligament into the joint space of one knee (3 mg/25 μl). The rats will be briefly anesthetized with isoflurane for this procedure.

On day 6, prior to receiving drug treatment, all animals will be submitted to a pre-test of the hind paws and pseudo-randomly assigned to treatment groups matched on the basis of the pain response of the lesioned hind paw (flexion-extension).

On days 14 and 21, the rats will receive drug treatment or vehicle (e.g., carrier alone) and will be evaluated for clinical and pain symptoms as described below:

### Clinical Symptoms Evaluation: Paw Use/Gait Score

The rats are placed in an open arena and their gait score is measured: the score is 0 if the rat stands bearing its weight equally on all four paws and appears to move normally. The score is 1 if the rat occasionally withdraws/lifts the lesioned paw; 2 if the rat often withdraws/lifts the lesioned paw and shows evidence of impaired use of the affected paw. The score is 3 if the rat permanently lifts the lesioned paw and movement is clearly affected.

### Pain Symptoms Evaluation

Each paw is evaluated by measuring the occurrence of vocalization following 5 consecutive flexions and 5 consecutive extensions. The total number of vocalizations to flexions and to extensions is recorded for the lesioned and non-lesioned paws.

### Tactile Allodynia Evaluation: Electronic Von Frey Test

The animal is placed under an inverted acrylic plastic box (18×11.5×14 cm) on a grid floor. The tip of an electronic von Frey probe is then applied with increasing force first to the non-lesioned and then the lesioned hindpaw and the force required to induce paw-withdrawal is automatically recorded. This procedure is carried out 3 times and the mean force per paw is calculated.

### Thermal Hyperalgesia Evaluation: Plantar Test

The apparatus consists of individual acrylic plastic boxes (18×11.5×14 cm) placed upon an elevated glass' floor. A rat is placed in the box and left free to habituate for 10 minutes. A mobile infrared radiant source is then focused first under the non-lesioned and then the lesioned hind paw and the paw-withdrawal latency is automatically recorded. In order to prevent tissue damage the heat source is automatically turned off after 45 seconds.

### Tactile Hyperalgesia Evaluation: Pinchmeter Test

The device consists of a pair of large blunt forceps equipped with 2 strain gauges connected to a modified electronic dynamometer. The tips of the forceps are placed around the hind paw. The force applied is incremented manually until the paw withdrawal response. The maximum force applied on the hind paw is automatically recorded and displayed by the dynamometer. In order to prevent tissue damage, the applied force is limited to a maximum of 1 kg. This procedure is carried out 3 times and the mean force is calculated.

Each test substance will be evaluated at 3 doses, administered p.o. b.i.d. from Day 7 to Day 20 and 60 minutes before the test on Day 14 and Day 21 and compared with a vehicle control group (29 administrations).

Tramadol (64 mg/kg) administered p.o. 60 minutes before the test on Day 14 will be used as analgesic comparison substance (except paw use) and as comparison substance on Day 21. To respect the number of dosing per rat, this group will receive distilled water as chronic administration.

The experiment will therefore include 17 groups. For the score and pain symptoms, inter-group comparison will be performed for the test substance using a Kruskall-Wallis Test, followed by Mann-Whitney U tests in case of significant group effect. For the reference substance, the treated group will be compared with vehicle control using Mann-Whitney U test.

For the electronic von Frey and plantar Tests, data with the test substance will be analyzed by comparing treated groups with vehicle control using ANOVA followed by post-hoc Dunnett's tests. Data with the reference substance will be analyzed using unpaired Student's t test.

### Terminal Blood Collection

### Plasma Sampling

At the completion of testing, all animals will be placed under isoflurane anesthesia and approximately 700 μl of blood will be collected by cardiac puncture using a sterile disposable syringe. The blood samples will be immediately transferred into pre-labeled tube containing K2-EDTA. After sealing each tube, the blood samples will be manually agitated and stored on ice until centrifugation (within 30 minutes of sampling). The samples will be centrifuged at +4° C., at 3000 g, for 10 minutes. The entire resultant plasma obtained will be immediately transferred to 2 suitably labeled polypropylene tubes (2 aliquots of approximately 150 μl) and flash frozen in liquid nitrogen or dry ice. The tubes will be stored upright at approximately −80° C.

Experimental treatment groups are described in Table 5. Compositions (“Comp.”) referred to in the treatment column correspond to those described in Table 1. The test or vehicle formulations will be administered as a repeated oral dose by gavage (b.i.d.). The vehicle will be sesame oil.

TABLE 5
Experimental treatment groups

| | | | | | Admin- |
|---|---|---|---|---|---|
| | | | Dose- | Concen- | istration |
| | Number of | | level | tration | Volume |
| Group | animals | Treatment | (mg/kg) | (mg/mL) | (mL/kg) |
| 1 | 10 males | Vehicle | — | — | 5 |
| 2 | 10 males | Comp. A | 5 | 1 | 5 |
| 3 | 10 males | Comp. A | 10 | 2 | 5 |
| 4 | 10 males | Comp. A | 50 | 10 | 5 |
| 5 | 10 males | Comp. C | 5 | 1 | 5 |
| 6 | 10 males | Comp. C | 10 | 2 | 5 |
| 7 | 10 males | Comp. C | 50 | 10 | 5 |
| 8 | 10 males | Comp. D | 5 | 1 | 5 |
| 9 | 10 males | Comp. D | 10 | 2 | 5 |
| 10 | 10 males | Comp. D | 50 | 10 | 5 |
| 11 | 10 males | Comp. E | 5 | 1 | 5 |
| 12 | 10 males | Comp. E | 10 | 2 | 5 |
| 13 | 10 males | Comp. E | 50 | 10 | 5 |
| 14 | 10 males | CBD-CBG-CBGA | 5 | 1 | 5 |
| | | extract | | | |
| 15 | 10 males | CBD-CBG-CBGA | 10 | 2 | 5 |
| | | extract | | | |
| 16 | 10 males | CBD-CBG-CBGA | 50 | 10 | 5 |
| | | extract | | | |
| 17 | 10 males | tramadol | 24 | 12.8 | 5 |

### Example 3. Study Using Telemetry in Conscious Rats

A study will be conducted to evaluate effects of hemp extracts on EEG by telemetry in conscious rats.

8 Wistar male rats (275 g-300 g) will be implanted with a telemetric transmitter in order to obtain 6 animals included in the study.

Each animal is dosed with the following treatments (n=6):
vehicle (e.g., carrier alone);
test item at 10 mg/kg;
test item at 30 mg/kg;
test item at 100 mg/kg. In this study the test item comprises Composition B of Table 1.

Treatments are separated by a one-week period of washout according to a cross-over study design.

Treatments will be single doses via oral gavage.

Two weeks (or more) before starting, animals will be implanted with a telemetric transmitter, with 2 electrodes placed in the cerebral cortex and 2 other electrodes in the muscle at the level of the neck. On the days of test, electrocorticogram (EEG) and electromyogram (EMG) will be recorded continuously. Body temperature and locomotor activity are also recorded.

The following parameters will be measured:
Sleep/awake cycles: Electrocorticogram is computed by the fast Fourier Transformation (FFT) for evaluation of the power spectrum of EEG rhythms. A computerized sleep scoring algorithm is used to determine the proportions of cumulated times of activity, quiet wake, slow wave sleep (SWS) and rapid eye movement (REM) as percentages per hour over the 24-hour periods.
Power of EEG rhythms (6, 9, a, 0, 'y) will be analyzed for each wake/sleep state separately.
Muscular tone: analyzed from the EMG
Body temperature.

### Example 4. Study Using the Elevated-Plus Maze Test in Rats

A study will be conducted to evaluate the effects of selected hemp extract substances in rats during the elevated-plus maze test.

The method detects anxiolytic activity. Rodents normally avoid open spaces (e.g., the open arms of an elevated plus-maze). Anxiolytics increase exploratory activity in the open arms, as indicated by increased time spent on the open arms and/or by increased open-arm entries. The maze comprises 4 arms of equal length and width (50 cm×10 cm) arranged in the form of a plus sign (+). Two opposite arms are enclosed by 40 cm high walls (closed arms). The 2 other arms have no walls (open arms). The maze is raised approximately 65 cm above the floor. A rat is placed in the center of the plus-maze and left to explore for 5 minutes. The number of entries into the open and closed arms and the time spent on the open arms are recorded. The percentage of open arm entries (open arm entries/total arm entries×100) is calculated.

10 rats are studied per group. The test will be performed blind.

Each hemp extract will be evaluated at 3 doses, administered p.o. b.i.d. from Day −5 to Day −1 and 120 minutes before the test on Day 0 (11 administrations) and compared with a vehicle control group (e.g., carrier alone).

Clobazam (64 mg/kg), administered p.o. 60 minutes before the test, will be used as reference substance.

Data with the test substance will be analyzed by comparing treated groups with vehicle control using one-way ANOVA followed by post-hoc Dunnett's tests. Data with the reference substance will be analyzed using unpaired Student's t tests.

### Plasma Sampling

At the completion of testing, all animals will be placed under isoflurane anesthesia and approximately 700 μl of blood will be collected by cardiac puncture using a sterile disposable syringe. The blood samples will be immediately transferred into pre-labeled tube containing K2-EDTA. After sealing each tube, the blood samples will be manually agitated and stored on ice until centrifugation (within 30 minutes of sampling). The samples will be centrifuged at +4° C., at 3000 g, for 10 minutes. The entire resultant plasma obtained will be immediately transferred to 2 suitably labeled polypropylene tubes (2 aliquots of approximately 150 μl) and flash frozen in liquid nitrogen or dry ice. The tubes will be stored upright at approximately −80° C.

Experimental treatment groups are described in Table 6. Compositions (“Comp.”) referred to in the treatment column correspond to those described in Table 1. The test or vehicle formulations will be administered as a repeated oral dose by gavage (b.i.d.). The vehicle will be sesame oil.

TABLE 6
Experimental treatment groups

| | | | Dose- | Concen- | Administration |
|---|---|---|---|---|---|
| | Number of | | level | tration | volume |
| Group | animals | Treatment | (mg/kg) | (mg/mL) | (mL/kg) |
| 1 | 10 males | control | — | — | 5 |
| | | substance | | | |
| 2 | 10 males | Comp. A | 5 | 1 | 5 |
| 3 | 10 males | Comp. A | 10 | 2 | 5 |
| 4 | 10 males | Comp. A | 50 | 10 | 5 |
| 5 | 10 males | Comp. B | 5 | 1 | 5 |
| 6 | 10 males | Comp. B | 10 | 2 | 5 |
| 7 | 10 males | Comp. B | 50 | 10 | 5 |
| 8 | 10 males | Comp. C | 5 | 1 | 5 |
| 9 | 10 males | Comp. C | 10 | 2 | 5 |
| 10 | 10 males | Comp. C | 50 | 10 | 5 |
| 11 | 10 males | clobazam | 64 | 12.8 | 5 |

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. A hemp extract comprising
20 mg/mL to 70 mg/mL of cannabidiol and
20 mg/mL to 70 mg/mL cannabidiolic acid;
wherein other cannabinoids are present at less than 5 mg/mL, individually.

2-7. (canceled)

8. A hemp extract comprising
20 mg/mL to 70 mg/mL of cannabigerol and
25 mg/mL to 70 mg/mL cannabigerolic acid;
wherein other cannabinoids are present at less than 5 mg/mL, individually.

9-14. (canceled)

15. A hemp extract comprising
20 mg/mL to 70 mg/mL of cannabidiolic acid;
20 mg/mL to 70 mg/mL cannabigerolic acid; and
1 mg/mL to 10 mg/mL of cannabigerol;
wherein any other cannabinoids are present at less than 5 mg/mL, individually.

16-21. (canceled)

22. A hemp extract comprising
10 mg/mL to 50 mg/mL of cannabidiol;
5 mg/mL to 35 mg/mL cannabidiolic acid;
5 mg/mL to 35 mg/mL of cannabidivarin;
3 mg/mL to 25 mg/mL cannabidivarinic acid; and
3 mg/mL to 20 mg/mL of cannabigerolic acid;
wherein other cannabinoids are present at less than 5 mg/mL, individually.

23-24. (canceled)

25. A hemp extract comprising
20 mg/mL to 40 mg/mL of cannabidiol;
10 mg/mL to 30 mg/mL cannabidiolic acid;
15 mg/mL to 30 mg/mL of cannabidivarin;
10 mg/mL to 20 mg/mL cannabidivarinic acid; and
8 mg/mL to 20 mg/mL of cannabigerolic acid;
wherein other cannabinoids are present at less than 5 mg/mL, individually.

26. A hemp extract comprising
5 mg/mL to 30 mg/mL of cannabidiol;
5 mg/mL to 30 mg/mL cannabidiolic acid;
5 mg/mL to 30 mg/mL of cannabidivarin; and
5 mg/mL to 30 mg/mL cannabidivarinic acid.

27-29. (canceled)

30. A hemp extract comprising
20 mg/mL to 60 mg/mL of cannabidiol;
20 mg/mL to 60 mg/mL of cannabidiolic acid; and
5 mg/mL to 30 mg/mL of cannabinol;
wherein other cannabinoids are present at less than 5 mg/mL, individually.

31-35. (canceled)

36. A hemp extract comprising:
20 mg/mL to 50 mg/mL of cannabidiol;
5 mg/mL to 25 mg/mL of cannabigerol; and
5 mg/mL to 25 mg/mL of cannabigerolic acid;
wherein other cannabinoids are present at less than 5 mg/mL, individually.

37-38. (canceled)

39. A hemp extract comprising:
5 mg/mL to 70 mg/mL of cannabidiolic acid;
5 mg/mL to 70 mg/mL of cannabigerolic acid; and
5 mg/mL to 70 mg/mL of cannabidivarinic acid,
wherein other cannabinoids are present at less than 5 mg/mL, individually.

40-53. (canceled)

54. A dosage form comprising the hemp extract of claim 1, wherein the dosage form comprises at least one of a pharmaceutically acceptable additive, a flavoring agent, a surfactant, and an adjuvant.

55-78. (canceled)

79. A method for treating chronic pain, anxiety, or inflammatory bowel disease in a subject in need thereof, the method comprising administering to the subject a therapeutically effective amount of the hemp extract of claim 1.

80-88. (canceled)

89. A method for treating chronic pain, anxiety, inflammatory bowel disease, or a sleep disorder in a subject in need thereof, the method comprising administering to the subject a therapeutically effective amount of the hemp extract of claim 8.

90. A method for treating chronic pain, anxiety, inflammatory bowel disease, or a sleep disorder in a subject in need thereof, the method comprising administering to the subject a therapeutically effective amount of the hemp extract of claim 15.

91. A method for treating chronic pain, anxiety, inflammatory bowel disease, or a sleep disorder in a subject in need thereof, the method comprising administering to the subject a therapeutically effective amount of the hemp extract of claim 22.

92. A method for treating chronic pain, anxiety, inflammatory bowel disease, or a sleep disorder in a subject in need thereof, the method comprising administering to the subject a therapeutically effective amount of the hemp extract of claim 25.

93. A method for treating chronic pain, anxiety, inflammatory bowel disease, or a sleep disorder in a subject in need thereof, the method comprising administering to the subject a therapeutically effective amount of the hemp extract of claim 26.

94. A method for treating chronic pain, anxiety, inflammatory bowel disease, or a sleep disorder in a subject in need thereof, the method comprising administering to the subject a therapeutically effective amount of the hemp extract of claim 30.

95. A method for treating chronic pain, anxiety, inflammatory bowel disease, or a sleep disorder in a subject in need thereof, the method comprising administering to the subject a therapeutically effective amount of the hemp extract of claim 36.

96. A method for treating chronic pain, anxiety, inflammatory bowel disease, or a sleep disorder in a subject in need thereof, the method comprising administering to the subject a therapeutically effective amount of the hemp extract of claim 39.
